(19) United States
(12) Patent Application Publication
(10) Pub. No.: US 2026/0028344 A1
(43) Pub. Date: Jan. 29, 2026

(54) CRYSTALLINE FORMS OF LANRAPLENIB AND METHODS OF MAKING AND USING THE SAME

(71) Applicant: Kronos Bio, Inc., San Mateo, CA (US)

(72) Inventors: Pasit Phiasivongsa, Hillsborough, CA (US); Xinnan Zhang, Los Altos, CA (US); Geoffrey Yeh, Milbrae, CA (US); Carim Van Beek, Foster City, CA (US); Chu-Yi Pang, New Taipei City (TW)

(21) Appl. No.: 19/231,299

(22) Filed: Jun. 6, 2025

Related U.S. Application Data
(63) Continuation of application No. PCT/US2023/082991, filed on Dec. 7, 2023.
(60) Provisional application No. 63/431,586, filed on Dec. 9, 2022.

(51) Int. Cl. C07D 487/04

(52) U.S. Cl. CPC C07D 487/04

(57) ABSTRACT

Described herein are high purity compositions of crystalline Compound I sesquisuccinate (Compound IA) and methods of preparing the same.

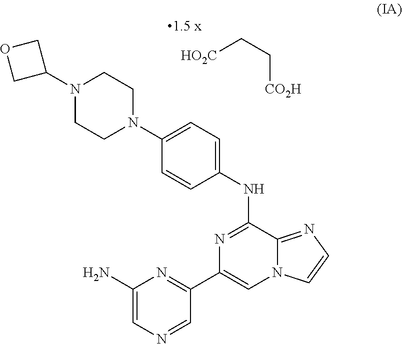 [SMILES: Nc1cncc(-c2cn3ccnc3c(Nc3ccc(N4CCN(C5COC5)CC4)cc3)n2)n1 ; O=C(O)CCC(=O)O]

### CROSS-REFERENCE TO RELATED APPLICATIONS

This application is a continuation of International Patent Application No. PCT/US2023/082991, filed Dec. 7, 2023, which claims priority to U.S. Prov. Patent App. No. 63/431,586, filed on Dec. 9, 2022, the disclosure of each of which is hereby incorporated herein by reference in its entirety.

### BACKGROUND

### Field

The present disclosure relates to crystalline forms and pharmaceutical compositions of compounds that inhibit Spleen Tyrosine Kinase (Syk) activity. The disclosure also relates to methods of preparing such crystalline forms and pharmaceutical compositions, and the use of such crystalline forms and pharmaceutical compositions in treating subjects with various diseases, including cancer and inflammatory conditions

### Background

The inhibition of Spleen Tyrosine Kinase (Syk) activity may be useful for treating certain types of cancer and autoimmune diseases. One such compound that has been found to inhibit Syk activity is represented as Compound I:

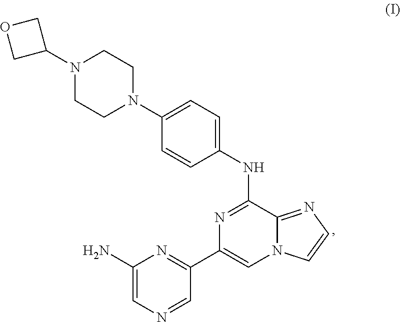 [SMILES: Nc1cncc(-c2cn3ccnc3c(Nc3ccc(N4CCN(C5COC5)CC4)cc3)n2)n1]

or a pharmaceutically acceptable salt thereof. This compound and its synthesis have been described in U.S. Pat. Nos. 8,440,667 and 8,455,493, which are hereby incorporated herein by reference in their entirety. U.S. Pat. No. 10,342,794 discloses several salt and polymorphic forms of Compound I, which is hereby incorporated herein by reference in its entirety.

Variations in the crystal structure of a pharmaceutical drug substance may affect the dissolution rate (which may affect bioavailability, etc.), manufacturability (such as ease of handling, ability to consistently prepare doses of known strength) and stability (such as thermal stability, shelf life, etc.) of a pharmaceutical drug product, particularly when formulated in a solid oral dosage form. In addition, the purity of pharmaceutical compositions comprising crystal forms of a drug substance can affect these properties and the suitability of a composition for pharmaceutical use. Accordingly, there is a need for high purity crystal structures and pharmaceutical compositions of the Compound I and processes for making the same.

### SUMMARY

Embodiments of the present application provide a crystalline (polymorphic) form of the sesquisuccinate salt or co-crystal of Compound I, shown below as Compound IA, having the structure:

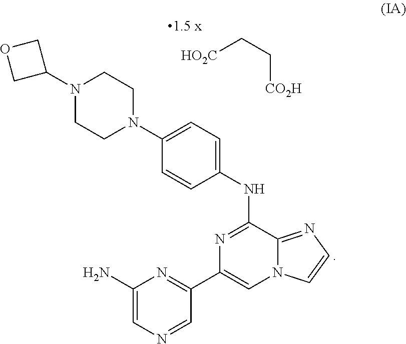 [SMILES: Nc1cncc(-c2cn3ccnc3c(Nc3ccc(N4CCN(C5COC5)CC4)cc3)n2)n1 ; O=C(O)CCC(=O)O]

It is to be understood that the Compound IA depicted as above includes the ionic form (such as, the cationic form of Compound I and the anionic form of succinic acid) as well as the neutral form (for example, in a co-crystal).

In some embodiments, provided herein is a composition comprising crystalline Compound IA, wherein the crystalline Compound IA exhibits an X-ray powder diffraction pattern comprising at least one characteristic peak, wherein said characteristic peak is selected from the group consisting of approximately 8.0, 8.3, 12.1, 16.5, 17.7, 21.8, 23.1, 24.5, 27.1, and 28.4 degrees 2θ, and wherein the crystalline Compound I sesquisuccinate in the composition comprises greater than about 75% of the total weight of the composition excluding pharmaceutically acceptable carriers.

In some embodiments, crystalline Compound I sesquisuccinate exhibits an X-ray powder diffraction pattern comprising at least three characteristic peaks, wherein said characteristic peaks are selected from the group consisting of approximately 8.0, 8.3, 12.1, 16.5, 17.7, 21.8, 23.1, 24.5, 27.1, and 28.4 degrees 2θ.

In some embodiments, an X-ray powder diffraction pattern of all Compound I in the composition does not contain a peak at approximately 19.9 degrees 2θ.

In some embodiments crystalline Compound I sesquisuccinate has unit cell dimensions: a=8.370 Å; b=11.991 Å; c=15.605 Å; α=70.25°. β=82.14°; and γ=77.70°.

In some embodiments, crystalline Compound I sesquisuccinate has an endotherm from about 188-192° C.

In some embodiments, crystalline Compound I sesquisuccinate has an endotherm at about 190° C.

In some embodiments, the composition comprises about 25 percent to about 30 percent by weight of succinate relative to the amount of Compound IA in the composition.

In some embodiments, the composition comprises about 26 percent to about 29 percent by weight of succinate relative to the amount of Compound IA in the composition.

In some embodiments, the composition comprises about 27 percent to about 29 percent by weight of succinate relative to the amount of Compound IA in the composition.

In some embodiments, the composition comprises about 28 percent to about 29 percent by weight of succinate relative to the amount of Compound IA in the composition.

In some embodiments, the total weight of crystalline Compound I sesquisuccinate in the composition comprises greater than about 75% excluding pharmaceutically acceptable carriers.

In some embodiments, the crystalline Compound I sesquisuccinate in the composition comprises greater than about 80% of the total weight of the composition excluding pharmaceutically acceptable carriers.

In some embodiments, the crystalline Compound I sesquisuccinate in the composition comprises greater than about 85% of the total weight of the composition excluding pharmaceutically acceptable carriers.

In some embodiments, the crystalline Compound I sesquisuccinate in the composition comprises greater than about 90% of the total weight of the composition excluding pharmaceutically acceptable carriers.

In some embodiments, the crystalline Compound I sesquisuccinate in the composition comprises greater than about 95% of the total weight of the composition excluding pharmaceutically acceptable carriers.

In some embodiments, the crystalline Compound I sesquisuccinate in the composition comprises greater than about 98% of the total weight of the composition excluding pharmaceutically acceptable carriers.

In some embodiments, the crystalline Compound I sesquisuccinate in the composition comprises greater than about 99% of the total weight of the composition excluding pharmaceutically acceptable carriers.

In some embodiments, the crystalline Compound I sesquisuccinate in the composition comprises greater than about 85% to about 100% of the total weight of the composition excluding pharmaceutically acceptable carriers.

In some embodiments, the crystalline Compound I sesquisuccinate in the composition comprises greater than about 90% to about 100% of the total weight of the composition excluding pharmaceutically acceptable carriers.

In some embodiments, the crystalline Compound I sesquisuccinate in the composition comprises greater than about 95% to about 99% of the total weight of the composition excluding pharmaceutically acceptable carriers.

Some embodiments include a process for preparing crystalline Compound I sesquisuccinate comprises the steps of:
(i) combining succinic acid and Compound I to form a crystallization mixture;
(ii) stirring the crystallization mixture; and
(iii) isolating a solid comprising Compound I sesquisuccinate from the crystallization mixture;
wherein at least 75% by weight of the solid isolated in step (iii) is crystalline Compound I sesquisuccinate, wherein the crystalline Compound I sesquisuccinate exhibits an X-ray powder diffraction pattern comprising at least three characteristic peaks, wherein said characteristic peaks are selected from the group consisting of approximately 8.0, 8.3, 12.1, 16.5, 17.7, 21.8, 23.1, 24.5, 27.1, and 28.4 degrees 2θ.

In some embodiments, a process for preparing crystalline Compound I sesquisuccinate is provided herein wherein the succinic acid is dissolved in a first solvent to form a first solution before combining said first solution with Compound I.

In some embodiments, a process for preparing crystalline Compound I sesquisuccinate is provided herein, wherein the first solvent is selected from the group consisting of tetrahydrofuran, 2-methyltetrahydrofuran, isopropyl alcohol, and acetone.

In some embodiments, a process for preparing crystalline Compound I sesquisuccinate is provided herein, wherein the first solvent is tetrahydrofuran.

In some embodiments, a process for preparing crystalline Compound I sesquisuccinate is provided herein, wherein the tetrahydrofuran comprises from about 1% to 10% by weight water.

In some embodiments, a process for preparing crystalline Compound I sesquisuccinate is provided herein, wherein the tetrahydrofuran comprises from about 1% to 5% by weight water.

In some embodiments, a process for preparing crystalline Compound I sesquisuccinate is provided herein, wherein the tetrahydrofuran comprises about 1.8% by weight water.

In some embodiments, a process for preparing crystalline Compound I sesquisuccinate is provided herein, wherein the first solution is heated to a temperature of from about 35 to 45° C.

In some embodiments, a process for preparing crystalline Compound I sesquisuccinate is provided herein, wherein the first solution is heated to a temperature of about 40° C.

In some embodiments, a process for preparing crystalline Compound I sesquisuccinate is provided herein, wherein the Compound I is dissolved in a second solvent to form a second solution before combining said second solution with succinic acid.

In some embodiments, a process for preparing crystalline Compound I sesquisuccinate is provided herein, wherein the second solvent is selected from the group consisting of tetrahydrofuran, 2-methyltetrahydrofuran, isopropyl alcohol, and acetone.

In some embodiments, a process for preparing crystalline Compound I sesquisuccinate is provided herein, wherein the solvent is tetrahydrofuran.

In some embodiments, a process for preparing crystalline Compound I sesquisuccinate is provided herein, wherein the tetrahydrofuran comprises from about 1% to 10% by weight water.

In some embodiments, a process for preparing crystalline Compound I sesquisuccinate is provided herein, wherein the tetrahydrofuran comprises from about 1% to 5% by weight water.

In some embodiments, a process for preparing crystalline Compound I sesquisuccinate is provided herein, wherein the tetrahydrofuran comprises about 1.8% by weight water.

In some embodiments, a process for preparing crystalline Compound I sesquisuccinate is provided herein, wherein the second solution is heated to a temperature of from about 35 to 45° C.

In some embodiments, a process for preparing crystalline Compound I sesquisuccinate is provided herein, wherein the second solution is heated to a temperature of about 40° C.

In some embodiments, a process for preparing crystalline Compound I sesquisuccinate is provided herein, wherein the second solution is added to the first solution to form the crystallization mixture.

In some embodiments, a process for preparing crystalline Compound I sesquisuccinate is provided herein, wherein the first solution is added to the second solution to form the crystallization mixture.

In some embodiments, a process for preparing crystalline Compound I sesquisuccinate is provided herein, wherein the first solution is added to the second solution over a period of about 10 to about 120 minutes.

In some embodiments, a process for preparing crystalline Compound I sesquisuccinate is provided herein, wherein the first solution is added to the second solution over a period of about 20 to about 60 minutes.

In some embodiments, a process for preparing crystalline Compound I sesquisuccinate is provided herein, wherein the first solution is added to the second solution over a period of about 20 to about 30 minutes.

In some embodiments, a process for preparing crystalline Compound I sesquisuccinate is provided herein, wherein the first solution is added to the second solution over a period of about 30 minutes.

In some embodiments, a process for preparing crystalline Compound I sesquisuccinate is provided herein, wherein the crystallization mixture is stirred at a temperature of from about 35 to 45° C.

In some embodiments, a process for preparing crystalline Compound I sesquisuccinate is provided herein, wherein the crystallization mixture is stirred at a temperature of about 40° C.

In some embodiments, a process for preparing crystalline Compound I sesquisuccinate is provided herein, wherein the crystallization mixture is stirred for from about 8 to about 36 hours.

In some embodiments, a process for preparing crystalline Compound I sesquisuccinate is provided herein, wherein the crystallization mixture is stirred for from about 12 to about 24 hours.

In some embodiments, a process for preparing crystalline Compound I sesquisuccinate is provided herein, wherein the crystallization mixture is stirred for from about 15 to about 21 hours.

In some embodiments, a process for preparing crystalline Compound I sesquisuccinate is provided herein, wherein the crystallization mixture is stirred for about 18 hours.

In some embodiments, a process for preparing crystalline Compound I sesquisuccinate is provided herein, wherein a sample of precipitated solid is removed from the crystallization mixture and analyzed for succinic acid content.

In some embodiments, a process for preparing crystalline Compound I sesquisuccinate is provided herein, if the sample of precipitated solid is less than 27% by weight succinate.

In some embodiments, a process for preparing crystalline Compound I sesquisuccinate is provided herein, wherein the Compound I is amorphous.

In some embodiments, a process for preparing crystalline Compound I sesquisuccinate is provided herein, wherein the Compound I in step (i) is a crystal form exhibiting an X-ray powder diffraction pattern comprising at least three characteristic peaks, wherein said characteristic peaks are selected from the group consisting of approximately 20.0, 18.0, 15.2, 10.8, 24.4, 20.8, 9.0, 19.4, and 17.3 degrees 2θ.

In some embodiments, a process for preparing crystalline Compound I sesquisuccinate is provided herein, wherein the Compound I in step (i) is a crystal form exhibiting an X-ray powder diffraction pattern comprising at least three characteristic peaks, wherein said characteristic peaks are selected from the group consisting of 19.4, 23.3, 22.5, 16.7, 24.3, 13.9, 18.8, 18.3 and 21.1 degrees 2θ.

In some embodiments, a process for preparing crystalline Compound I sesquisuccinate is provided herein, wherein the Compound I in step (i) is a crystal form exhibiting an X-ray powder diffraction pattern comprising at least three characteristic peaks, wherein said characteristic peaks are selected from the group consisting of 19.7, 22.7, 17.7, 14.0, 20.0, 8.6, 14.9, 21.3 and 17.2 degrees 2θ.

In some embodiments, a process for preparing crystalline Compound I sesquisuccinate is provided herein, wherein if the succinic acid content in the sample of step (iii) is below about 27% by weight of the sample of precipitated solid, said crystallization mixture is stirred for an additional period of time.

In some embodiments, a process for preparing crystalline Compound I sesquisuccinate is provided herein, wherein greater than about 90% by weight of the solid isolated in step (iii) is crystalline Compound I sesquisuccinate.

In some embodiments, a process for preparing crystalline Compound I sesquisuccinate is provided herein, wherein greater than about 95% by weight of the solid isolated in step (iii) is crystalline Compound I sesquisuccinate.

In some embodiments, a process for preparing crystalline Compound I sesquisuccinate is provided herein, wherein greater than about 98% by weight of the solid isolated in step (iii) is crystalline Compound I sesquisuccinate.

In some embodiments, a process for preparing crystalline Compound I sesquisuccinate is provided herein, greater than about 99% by weight of the solid isolated in step (iii) is crystalline Compound I sesquisuccinate.

In some embodiments, a process for preparing crystalline Compound I sesquisuccinate is provided herein, wherein about 85% to about 99% by weight of the solid isolated in step (iii) is crystalline Compound I sesquisuccinate.

In some embodiments, a process for preparing crystalline Compound I sesquisuccinate is provided herein, wherein about 90% to about 99% by weight of the solid isolated in step (iii) is crystalline Compound I sesquisuccinate.

In some embodiments, a process for preparing crystalline Compound I sesquisuccinate is provided herein, wherein about 95% to about 99% by weight of the solid isolated in step (iii) is crystalline Compound I sesquisuccinate.

In some embodiments, a process for preparing crystalline Compound I sesquisuccinate is provided herein, wherein the molar ratio of Compound I to succinic acid combined in step (i) is from about 1:0.5 to about 1:5.

In some embodiments, a process for preparing crystalline Compound I sesquisuccinate is provided herein, wherein the molar ratio of Compound I to succinic acid combined step (i) is from about 1:1 to about 1:3.

In some embodiments, a process for preparing crystalline Compound I sesquisuccinate is provided herein, wherein the molar ratio of Compound I to succinic acid combined in step (i) is from about 1:1.5 to about 1:2.5.

In some embodiments, a process for preparing crystalline Compound I sesquisuccinate is provided herein, wherein the molar ratio of Compound I to succinic acid combined in step (i) is about 1:1.5.

In some embodiments, a process for preparing crystalline Compound I sesquisuccinate is provided herein, wherein the molar ratio of Compound I to succinic acid combined in step (i) is about 1:1.8.

In some embodiments, a process for preparing crystalline Compound I sesquisuccinate is provided herein, wherein the molar ratio of Compound I to succinic acid combined in step (i) is about 1:2.

In some embodiments, a process for preparing crystalline Compound I sesquisuccinate is provided herein, further comprising the step of filtering the crystallization mixture to recover wet Compound I sesquisuccinate.

In some embodiments, a process for preparing crystalline Compound I sesquisuccinate is provided herein, wherein the wet Compound I sesquisuccinate is dried at a temperature of about 50° C. to about 70° C.

In some embodiments, a process for preparing crystalline Compound I sesquisuccinate is provided herein, wherein an X-ray powder diffraction pattern of all isolated solid Compound I does not exhibit a peak at approximately 19.9 degrees 2θ.

In some embodiments, a composition prepared by the processes provided herein is disclosed.

In some embodiments, the composition may further comprise a pharmaceutically acceptable excipient.

In some embodiments, a process for preparing substantially pure crystalline Compound I sesquisuccinate from a composition comprising one or more crystalline or amorphous forms of Compound I sesquisuccinate and optionally one or more crystalline or amorphous forms of Compound I is provided herein, said process comprising the steps of:
(i) treating said composition with aqueous sulfuric acid to form Compound I sulfate; and
(ii) treating said Compound I sulfate with a base to form a first solution of Compound I; and
(iii) filtering said solution of Compound I to remove solids to form a second solution of Compound I.

In some embodiments, a process for preparing crystalline Compound I sesquisuccinate is provided herein, wherein isopropanol is added to said second solution of Compound I to form a slurry of Compound I.

In some embodiments, a process for preparing crystalline Compound I sesquisuccinate is provided herein, wherein the base is selected from sodium carbonate, lithium carbonate, potassium carbonate, and cesium carbonate.

In some embodiments, a process for preparing crystalline Compound I sesquisuccinate is provided herein, wherein said process further comprises the steps of:
(i) combining succinic acid and Compound I to form a crystallization mixture;
(ii) stirring the crystallization mixture; and
(iii) isolating solid Compound I sesquisuccinate from the crystallization mixture.

In some embodiments, a process for preparing crystalline Compound I sesquisuccinate is provided herein, wherein said crystalline Compound I sesquisuccinate has X-ray powder diffraction pattern that does not exhibit a peak at 19.9 degrees 2θ.

In some embodiments provided herein is a composition comprising crystalline Compound I sesquisuccinate, wherein the crystalline Compound I sesquisuccinate exhibits an X-ray powder diffraction pattern comprising at least three characteristic peaks, wherein said characteristic peaks are selected from the group consisting of approximately 8.0, 8.3, 12.1, 16.5, 17.7, 21.8, 23.1, 24.5, 27.1, and 28.4 degrees 2θ, and
wherein the total weight of crystalline Compound I sesquisuccinate in the composition is greater than about 75%.

### BRIEF DESCRIPTION OF THE DRAWINGS

FIG. 1. is a simulated XRPD pattern generated from the single crystal data for crystalline Compound IA.

FIG. 2 is an X-Ray Powder Diffraction (XRPD) pattern of crystalline Compound IA prepared with known methods.

FIG. 3 is an XRPD pattern for crystalline Compound I free base.

FIG. 4. is an XRPD pattern showing conversion of impure bulk crystalline Compound IA into the corresponding free base and subsequent processing according to the methods described herein to form pure crystalline Compound IA.

FIG. 5 illustrates an exemplary XRPD pattern of Compound I freebase polymorphic form I.

FIG. 6 illustrates an exemplary XRPD pattern of Compound I freebase polymorphic form V.

FIG. 7 illustrates an exemplary XRPD pattern of Compound I freebase polymorphic form XIV.

### DETAILED DESCRIPTION

In general, crystalline free base forms of an active pharmaceutical ingredient (API) are more physically stable than their ionized salts and cocrystal or coformer, with a similar hydrogen bonding in hydrate and solvate. However, for immediate release formulations, with a rapid release of API, salts and cocrystals are better suited for their multi-fold increase in dissolution rate and solubility for APIs with biopharmaceutical classification system (BCS) class II and IV. Lanraplenib, a BCS class II molecule, has low solubility in physiological conditions. Lanraplenib, a second generation SYK inhibitor with improved target specificity, is being developed to treat patients with acute myeloid leukemia (AML) as an immediate release (IR) formulated oral dosage tablet, for once daily administration, to achieve rapid exposure and engagement of the spleen tyrosine kinase (SYK). The safety and efficacy of the IR formulation of lanraplenib is being established in Phase 1/2 in AML, to establish pharmacokinetic (PK) profile and doses where target SYK engagement or pharmacodynamic (PD) effect is significant and clinically efficacious.

Preparation of some salts and cocrystals may result in APIs that do not have the desired purity. Additionally, salt or cocrystal disproportionation (a conversion from the ionized state for salt or un-ionized coformed cocrystal to the neutral free base form) in solid formulation is a potential concern during manufacturing or storage of products containing a salt or cocrystal (as the API) due to the negative ramifications of reduced (either reduction or variability) product performance. It was discovered that manufacturing of crystalline Compound IA using known processes exhibited some of these problems. Accordingly, provided herein is a scalable manufacturing process to produce a pure crystalline Compound IA.

### Definitions

As used in the present disclosure, the following words and phrases are generally intended to have the meanings as set forth below, except to the extent that the context in which they are used indicates otherwise.

The term “polymorph” refers to different crystal structures of a crystalline compound. The different polymorphs may result from differences in crystal packing (packing polymorphism) or differences in packing between different conformers of the same molecule (conformational polymorphism). It is understood that any polymorph of Compound I, or a pharmaceutically acceptable salt or co-crystal thereof, used in the treatment of a disease or condition as described herein, while possibly providing varied properties, including pharmacokinetic properties, once absorbed into the subject, results in the Compound I, such that the use of Compound I encompasses the use of any polymorph of Compound I, respectively, or a pharmaceutically acceptable salt or co-crystal thereof.

The term “cocrystal” refers to solids that are crystalline single phase materials composed of two or more different molecular and/or ionic compounds generally in a stoichiometric ratio which are neither solvates nor simple salts.

The term “solvate” refers to a complex formed by the combining of a Compound IA and a solvent.

The term “hydrate” refers to the complex formed by the combining of a Compound IA and water.

### Compounds

Provided are pharmaceutically acceptable salts or co-crystals of Compound I:

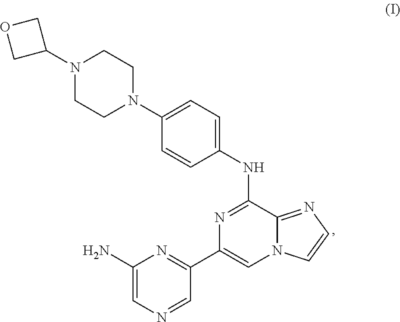 [SMILES: Nc1cncc(-c2cn3ccnc3c(Nc3ccc(N4CCN(C5COC5)CC4)cc3)n2)n1]

In some embodiments, the pharmaceutically acceptable salt or cocrystal is a sesquisuccinate salt or cocrystal. The sesquisuccinate salt or cocrystal of Compound I may be depicted herein in various ways. For example, in one variant, the sesquisuccinate salt or cocrystal of the Compound I may be depicted as Compound IA:

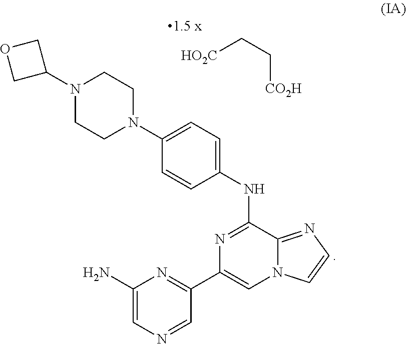 [SMILES: Nc1cncc(-c2cn3ccnc3c(Nc3ccc(N4CCN(C5COC5)CC4)cc3)n2)n1 ; O=C(O)CCC(=O)O]

Some embodiments provide a crystalline form of the sesquisuccinate salt or cocrystal of Compound I.

### Single Crystal X-ray Diffraction

The structure of crystalline Compound IA described herein was identified by single crystal X-ray analysis. The freebase starting material was formed into a sesquisuccinate co-crystal by the addition of succinic acid in tetrahydrofuran, followed by filtration and slow cooling. A yellow plate-like single crystal with high diffraction quality was immersed in Paratone-N, an oil based cryoprotectant. The crystal was mounted on a mylar loop in a random orientation and immersed in a stream of liquid nitrogen at 200K. The X-ray intensity data were measured on a Bruker D8 VENTURE (IμS microfocus X-ray source, Cu Kα, λ=1.54178 Å, PHOTON CMOS detector) diffractometer. The frames were integrated with the Bruker SAINT software package. The integration of the data using a triclinic unit cell yielded a total of 55058 reflections to a maximum θ angle of 67.679° (0.83 Å resolution), of which 5264 were independent (completeness=99.9%, Rᵢₙₜ=2.98%) and were greater than 2σ(F²). The final cell constants of α=8.370(3) Å, b=11.991(5) Å, c=15.605(3) Å, α=70.25(3)°, β=82.14(2)°, γ=77.70(3°), cell volume=1436.7(9) Å³, are based upon the refinement of the XYZ-centroids of 9946 reflections above 20 σ(I) with 5.417°<θ<79.494°. Data were corrected for absorption effects using the Multi-Scan method (SADABS). The absorption coefficient μ of this material is 0.879 mm⁻¹ at this wavelength (1.54178 Å). The calculated minimum and maximum transmission coefficients (based on crystal size) are 0.6805 and 0.7538.

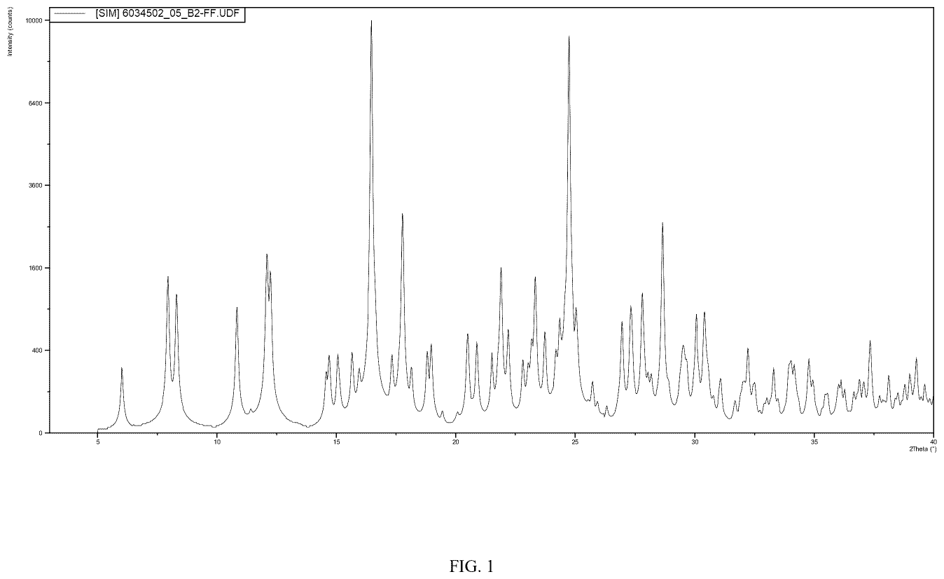

Using the single crystal X-ray data, a simulated XRPD pattern (FIG. 1) was generated using Mercury 2020.2.0. X'Pert HighScore Plus 3.0 was used for the identification and characterization of the simulated peaks, as shown in Table 1.

The structure for crystalline Compound IA was solved with the ShelXL structure solution program using Intrinsic Phasing and refined with ShelXL (Version 2014/7) refinement package using full-matrix least-squares on F² using the space group P1̅, with Z=2 for the formula unit, C₂₉H₃₄N₉O₇). Sheldrick, G. M. SHELXTL-2014, Crystallographic Computing System; Bruker Analytical X-Ray Instruments: Madison, WI, 2014; (b) Sheldrick, G. M. A Short History of SHELX, Acta Cryst. 2008, A64, 112 All non-hydrogen atoms were refined anisotropically. The positions of the hydrogen atoms connected to carbon atoms were calculated geometrically and refined using the riding model. The final anisotropic full-matrix least-squares refinement on F² with 418 variables converged at R₁=3.30%, for the observed data and wR₂=9.01% for all data. The goodness-of-fit was 1.044. The largest peak in the final difference electron density map was 0.192 e⁻/Å³ and the largest hole was −0.238 e⁻/Å³. Based on the final model, the calculated density is 1.435 g/cm³ and F (000), 654 e⁻. Table 1 lists the peak values for the calculated XRPD pattern of crystalline Compound IA.

TABLE 1
Peak Values of Calculated XRPD Patterns
of Crystalline Compound IA

| Pos. | Relative | d-spacing |
|---|---|---|
| [°2Th.] | Intensity | [Å] |
| 6.0 | 0.025 | 14.7 |
| 8.0 | 0.11 | 11.1 |
| 8.3 | 0.11 | 10.6 |
| 10.8 | 0.074 | 8.2 |
| 12.1 | 0.15 | 7.3 |
| 12.3 | 0.12 | 7.2 |
| 14.6 | 0.013 | 6.1 |
| 14.7 | 0.028 | 6.0 |
| 15.1 | 0.028 | 5.9 |
| 15.7 | 0.030 | 5.7 |
| 15.9 | 0.015 | 5.6 |
| 16.5 | 1 | 5.4 |
| 17.3 | 0.028 | 5.1 |
| 17.8 | 0.28 | 5.0 |
| 18.2 | 0.020 | 4.9 |
| 18.8 | 0.031 | 4.7 |
| 19.0 | 0.037 | 4.7 |
| 20.5 | 0.058 | 4.3 |
| 20.9 | 0.038 | 4.2 |
| 21.5 | 0.038 | 4.1 |
| 21.9 | 0.13 | 4.1 |
| 22.2 | 0.050 | 4.0 |
| 22.8 | 0.025 | 3.9 |
| 23.3 | 0.11 | 3.8 |
| 23.7 | 0.060 | 3.7 |
| 24.2 | 0.024 | 3.7 |
| 24.4 | 0.045 | 3.7 |
| 24.7 | 0.74 | 3.6 |
| 25.1 | 0.071 | 3.6 |
| 25.7 | 0.012 | 3.5 |
| 27.0 | 0.058 | 3.3 |
| 27.3 | 0.093 | 3.3 |
| 27.8 | 0.11 | 3.2 |
| 28.2 | 0.016 | 3.2 |
| 28.7 | 0.21 | 3.1 |
| 29.5 | 0.044 | 3.0 |
| 29.7 | 0.018 | 3.0 |
| 30.1 | 0.066 | 3.0 |

Based on the structural solution, it was unexpectedly discovered that the carboxylate protons on the succinic acid moieties do not transfer to Compound I. Thus, the proton positions for both the full succinic acid and the half succinic acid in the unit cell unexpectedly identify Compound IA as a cocrystal.

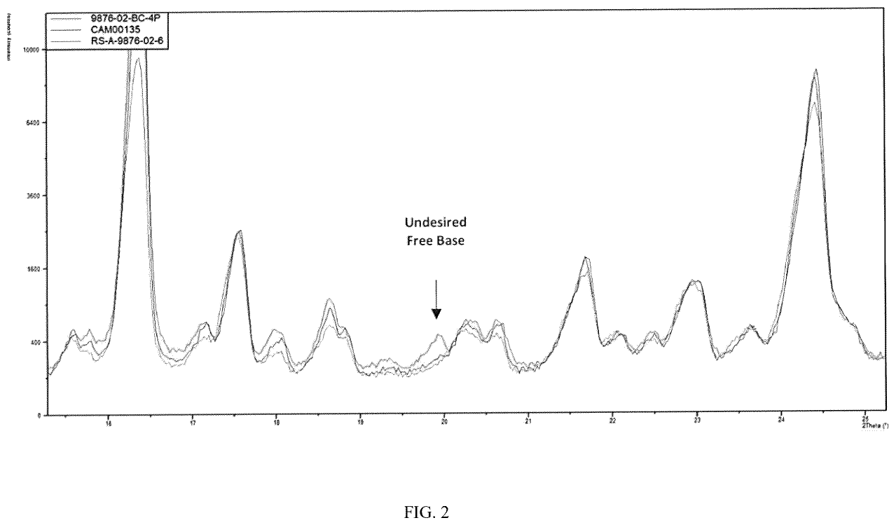

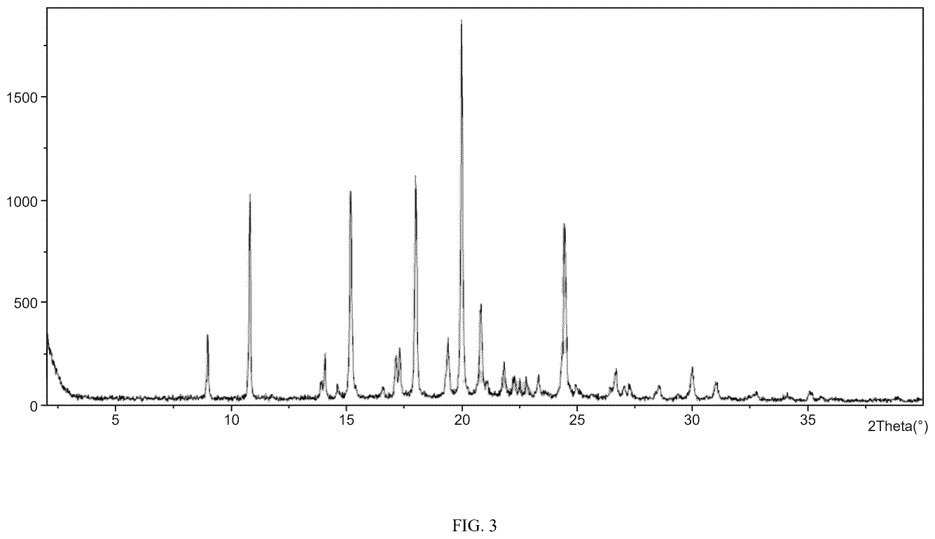

It was discovered that prior processes for preparing crystalline Compound IA do not produce pure crystalline Compound IA having only the XRPD peaks of Table 1. Rather, these prior processes result in some Compound I free base present in the product which can be identified by the presence of an XRPD peak at approximately 19.9 degrees 2θ. The product obtained with the prior processes is shown as FIG. 2. The XRPD peak at approximately 19.9 degrees 2θ is a characteristic peak of crystalline Compound I free base, as evidenced by the XRPD of the free base (FIG. 3). However, the processes described herein for preparing crystalline Compound IA result in compositions substantially free of Compound I free base.

Compositions of a Crystalline from of Compound I Sesquisuccinate

Provided herein are compositions comprising a crystalline form of Compound I sesquisuccinate (Compound IA), wherein, the crystalline form exhibits an X-ray powder diffraction pattern having at least one characteristic peak selected from the group consisting of approximately 8.0, 8.3, 12.1, 16.5, 17.7, 21.8, 23.1, 24.5, 27.1, and 28.4 degrees 2θ. In some embodiments, the crystalline form exhibits an X-ray powder diffraction pattern having at least three characteristic peaks selected from the group consisting of approximately 8.0, 8.3, 12.1, 16.5, 17.7, 21.8, 23.1, 24.5, 27.1, and 28.4 degrees 2θ. Some embodiments include compositions having an X-ray powder diffraction pattern that does not exhibit a peak at 19.9 degrees 2θ.

In some embodiments, the crystalline Compound I sesquisuccinate (Compound IA) in the composition comprises greater than about 75% by weight of the composition. In some embodiments, the crystalline Compound I sesquisuccinate (Compound IA) in the composition comprises greater than about 75% by weight of the composition excluding pharmaceutically acceptable carriers.

In some embodiments, the total weight of crystalline Compound IA in the composition is greater than about 80%, 81%, 82%, 83%, 84%, 85%, 86%, 87%, 88%, 89%, 90%, 91%, 92%, 93%, 94%, 95%, 96%, 97%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, or 99.9% by weight. In some embodiments, the total weight crystalline Compound IA in the composition is greater than about 80%, 81%, 82%, 83%, 84%, 85%, 86%, 87%, 88%, 89%, 90%, 91%, 92%, 93%, 94%, 95%, 96%, 97%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, or 99.9% by weight excluding pharmaceutically acceptable carriers.

In some embodiments, crystalline Compound IA has unit cell dimensions approximately measuring a=8.370 Å; b=11.991 Å; c=15.605 Å; α=70.25°. β=82.140; and γ=77.70°.

In some embodiments, crystalline Compound IA has an endotherm from about 185-195° C., for example crystalline Compound IA may have an endotherm of about 185° C., about 186° C., about 187° C., about 188° C., about 189° C., about 190° C., about 191° C., about 192° C., about 193° C., about 194° C., about 195° C., or within a range defined by any of the aforementioned values. For example, crystalline Compound IA may have an endotherm of about 190° C., or between about 188° C., about 192° C.

In some embodiments, the total weight of crystalline Compound IA in the composition is about 85% to about 100%, about 90% to about 100%, or about 95% to about 99%. In some embodiments, the total weight of crystalline Compound IA in the composition is about 85% to about 100% excluding pharmaceutically acceptable carriers.

In some embodiments, the composition comprises no more than 20% by weight of one or more impurities, for example 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6% 0.5%, 0.4%, 0.3%, 0.2% 0.1% or 0%, or within a range defined by any two of the aforementioned values. In some embodiments, the composition comprises no more than 10% by weight of one or more impurities, for example 10%, 9%, 8%, 7%, 6%, 5%, 4%, 3%, 2%, 1%, 0.9%, 0.8%, 0.7%, 0.6% 0.5%, 0.4%, 0.3%, 0.2% 0.1% or 0%, or within a range defined by any two of the aforementioned values. In some embodiments, the composition comprises no more than 5% by weight of one or more impurities, for example 5%, 4%, 3%, 2%, 1%, 0.9%, 0.8%, 0.7%, 0.6% 0.5%, 0.4%, 0.3%, 0.2% 0.1% or 0%, or within a range defined by any two of the aforementioned values. In some embodiments, the composition comprises no more than 2% by weight of one or more impurities, for example 2%, 1%, 0.9%, 0.8%, 0.7%, 0.6% 0.5%, 0.4%, 0.3%, 0.2% 0.1% or 0%, or within a range defined by any two of the aforementioned values. In some embodiments, the composition comprises no more than 1% by weight of one or more impurities, for example 1%, 0.9%, 0.8%, 0.7%, 0.6% 0.5%, 0.4%, 0.3%, 0.2% 0.1%, or 0%.

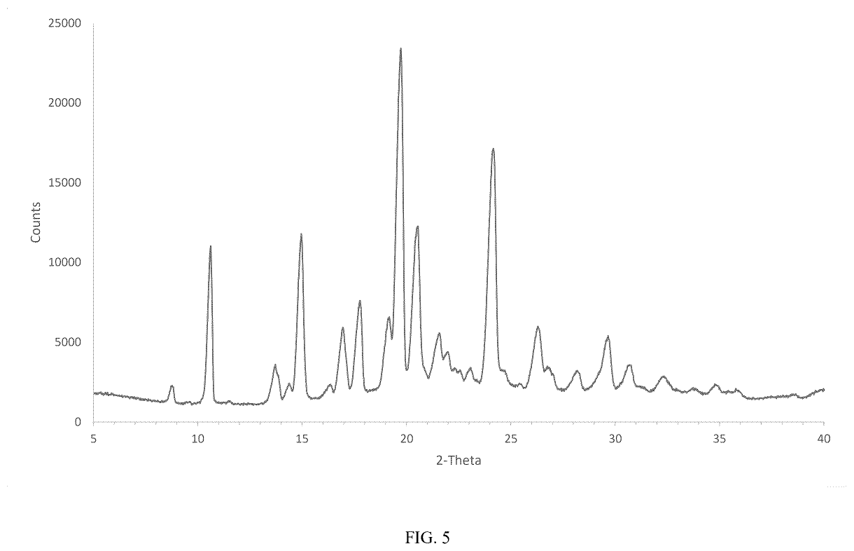

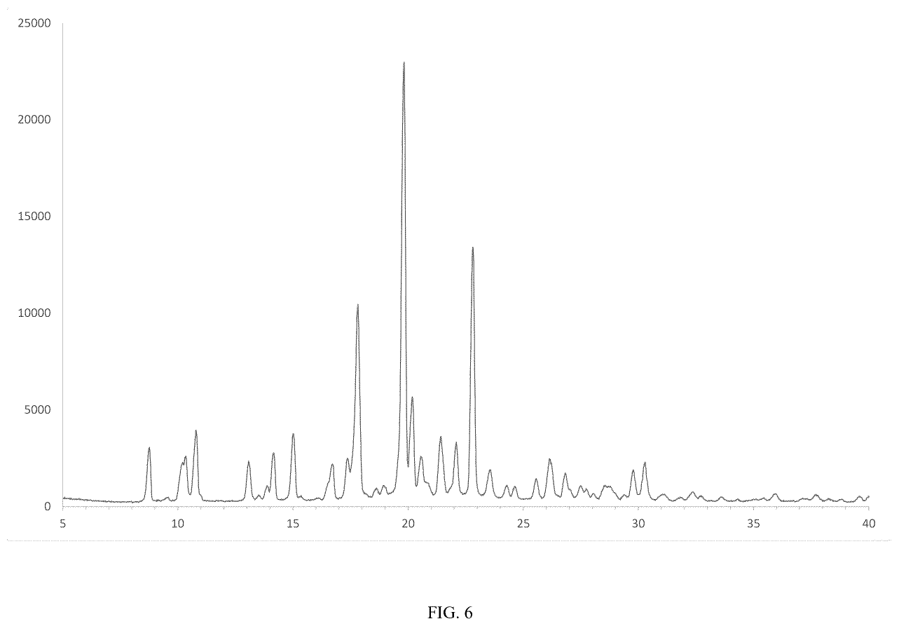

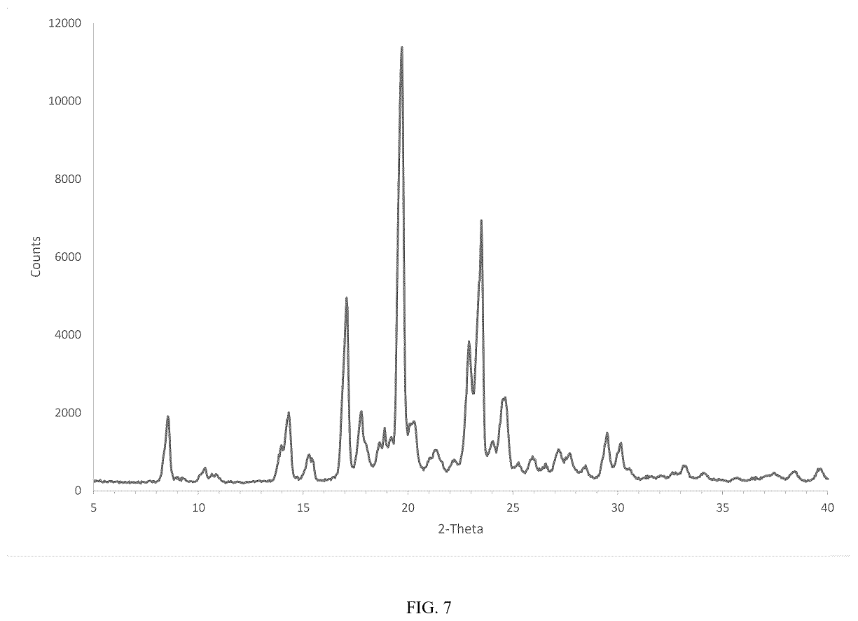

In some embodiments, the one or more impurities are selected from the group consisting of: Compound I freebase; Compound I monosuccinate; Compound I disuccinate; Compound I trisuccinate; Compound I tetrasuccinate; Compound I pentasuccinate; crystalline Compound IA wherein the compound does not exhibit at least three characteristic peaks on X-ray powder diffraction, wherein said characteristic peaks are selected from the group consisting of approximately 8.0, 8.3, 12.1, 16.5, 17.7, 21.8, 23.1, 24.5, 27.1, and 28.4 degrees 2θ. In some embodiments, the Compound I freebase impurity is in amorphous form. In some embodiments, the Compound I freebase impurity is in polymorphic form I, exhibiting at least three characteristic peaks selected from the group consisting of approximately 10.6, 15.0, 19.7, 20.5, and 24.2 degrees 2θ. An exemplary XRPD pattern of Compound I freebase polymorphic form I is shown in FIG. 5. In some embodiments, the Compound I freebase impurity is in polymorphic form V, exhibiting at least three characteristic peaks selected from the group consisting of approximately 10.8, 17.8, 19.8, 20.2, and 22.8 degrees 2θ. An exemplary XRPD pattern of Compound I freebase polymorphic form V is shown in FIG. 6. In some embodiments, the Compound I freebase impurity is in polymorphic form XIV, exhibiting at least three characteristic peaks selected from the group consisting of approximately 17.1, 19.7, 22.9, 23.5, and 24.6 degrees 2θ. An exemplary XRPD pattern of Compound I freebase polymorphic form XIV is shown in FIG. 7.

In some embodiments, the composition comprises no more than 20% by weight of Compound free base (i.e., Compound I), for example 20%, 19%, 18%, 17%, 16%, 15%, 14%, 13%, 12%, 11%, 10%, 9%, 8%, 7%, 6%, 5%, 4%, 3%, 2%, 1.9%, 1.8%, 1.7%, 1.6% 1.5%, 1.4%, 1.3%, 1.2% 1.1%, 1%, 0.9%, 0.8%, 0.7%, 0.6% 0.5%, 0.4%, 0.3%, 0.2% 0.1% or 0%, or within a range defined by any two of the aforementioned values. In some embodiments, the composition comprises no more than 10% by weight of Compound I, for example 10%, 9%, 8%, 7%, 6%, 5%, 4%, 3%, 2%, 1%, 0.9%, 0.8%, 0.7%, 0.6% 0.5%, 0.4%, 0.3%, 0.2% 0.1% or 0%, or within a range defined by any two of the aforementioned values. In some embodiments, the composition comprises no more than 5% by weight of free base Compound I, for example 5%, 4%, 3%, 2%, 1%, 0.9%, 0.8%, 0.7%, 0.6% 0.5%, 0.4%, 0.3%, 0.2% 0.1% or 0%, or within a range defined by any two of the aforementioned values. In some embodiments, the composition comprises no more than 2% by weight of free base Compound I, for example 2%, 1%, 0.9%, 0.8%, 0.7%, 0.6% 0.5%, 0.4%, 0.3%, 0.2% 0.1% or 0%, or within a range defined by any two of the aforementioned values. In some embodiments, the composition comprises no more than 1% by weight of free base Compound I, for example 1%, 0.9%, 0.8%, 0.7%, 0.6% 0.5%, 0.4%, 0.3%, 0.2% 0.1%, or 0%.

In some embodiments, the composition further comprises a pharmaceutically acceptable carrier.

In some embodiments, the composition comprises about 25 percent to about 30 percent by weight of succinate relative to the amount of Compound IA in the composition. In some embodiments, the composition described herein comprises about 25 percent to about 30 percent by weight of succinate relative to the amount of Compound IA in the composition. In some embodiments, the composition described herein comprises about 27.9 percent by weight of succinate relative to the amount of Compound IA in the composition. In other embodiments, the composition described herein comprises about 25.6 percent to about 27.9 percent by weight relative to the amount of Compound IA in the composition. In other embodiments, the composition described herein comprises about 27.9 percent to about 30 percent by weight of succinate relative to the amount of Compound IA in the composition. In some embodiments, the composition described herein comprises about 27.9 percent to about 28.5 percent by weight of succinate relative to the amount of Compound IA in the composition. In other embodiments, the composition described herein comprises about 25 percent to about 29 percent by weight of succinate relative to the amount of Compound IA in the composition. In other embodiments, the composition described herein comprises about 29 percent to about 30 percent by weight of succinate relative to the amount of Compound IA in the composition. In other embodiments, the composition described herein comprises about 27.9 percent to about 29.1 percent by weight of succinate. In other embodiments, the composition described herein comprises about 28 percent to about 30 percent by weight succinate relative to the amount of Compound IA in the composition. In other embodiments, the composition described herein comprises about 28.2 percent by weight succinate relative to the amount of Compound IA in the composition.

In some embodiments, the percent succinate content relative to all forms of Compound I may be approximately 25%, 25.1%, 25.2%, 25.3%, 25.4%, 25.5%, 25.6%, 25.7%, 25.8%, 25.9%, 26%, 26.1%, 26.2%, 26.3%, 26.4%, 26.5%, 26.6%, 26.7%, 26.8%, 26.9%, 27%, 27.1%, 27.2%, 27.3%, 27.4%, 27.5%, 27.6%, 27.7%, 27.8%, 27.9%, 28%, 28.1%, 28.2%, 28.3%, 28.4%, 28.5%, 28.6%, 28.7%, 28.8%, 28.9%, 29%, 29.1%, 29.2%, 29.3%, 29.4%, 29.5%, 29.6%, 29.7%, 29.8%, 29.9%, 30%, or within a range approximately defined by any of the two aforementioned values. For example, the percent succinate content relative to all forms of Compound I may be approximately 28% by weight to 30% by weight, or 27.9% by weight to 28.5% by weight, or 25% by weight to 30% by weight.

In some embodiments, the percent water content by weight for the crystalline Compound IA described herein may be approximately 0.005%, 0.01%, 0.015%, 0.02%, 0.025%, 0.03%, 0.035%, 0.04%, 0.045%, 0.05%, 0.055%, 0.06%, 0.065%, 0.07%, 0.075%, 0.08%, 0.081%, 0.082%, 0.083%, 0.084%, 0.085%, 0.086%, 0.087%, 0.088%, 0.089%, 0.090%, 0.091%, 0.092%, 0.093%, 0.094%, 0.095%, 0.096%, 0.097%, 0.098%, 0.099%, 0.10%, 0.101%, 0.102%, 0.103%, 0.104%, 0.105%, 0.106%, 0.107%, 0.108%, 0.109%, 0.11%, 0.115%, 0.12%, 0.125%, 0.13%, 0.135%, 0.14%, 0.145%, 0.150%, 0.151%, 0.152%, 0.153%, 0.154%, 0.155%, 0.156%, 0.157%, 0.158%, 0.159%, 0.160%, 0.161%, 0.162%, 0.163%, 0.164%, 0.165%, 0.166%, 0.167%, 0.168%, 0.169%, 0.170%, 0.171%, 0.172%, 0.173%, 0.174%, 0.175%, 0.176%, 0.177%, 0.178%, 0.179%, 0.180%, 0.181%, 0.182%, 0.183%, 0.184%, 0.185%, 0.186%, 0.187%, 0.188%, 0.189%, 0.190%, 0.191%, 0.192%, 0.193%, 0.194%, 0.195%, 0.196%, 0.197%, 0.198%, 0.199%, 0.200%, or within a range defined approximately by any two of the aforementioned values. For example, the range of the percent water content by weight for crystalline Compound IA may be approximately 0.005% by weight to 0.18% by weight, 0.005% by weight to 0.20% by weight, 0.09% by weight to 0.18% by weight, from 0.005% to 0.30% by weight.

### Crystallization and Conversion Between the Free Base Form of Formula I and Formula IA

Provided herein is a process for preparing crystalline Compound I sesquisuccinate (Compound IA), said process comprising the steps of: combining succinic acid and Compound I to form a crystallization mixture; stirring the crystallization mixture; and isolating a solid comprising Compound I sesquisuccinate from the crystallization mixture.

In some embodiments, a process for generating substantially pure crystalline Compound IA, e.g., the crystalline form described herein, from bulk composition of one or more crystalline or amorphous forms Compound IA and additionally one or more crystalline or amorphous forms of Compound I may comprise treating the entire bulk composition with aqueous sulfuric acid, which forms Compound I sulfate, followed by treating the Compound I sulfate with a base to form a first solution of Compound I (i.e., the free base), followed by filtering the first solution to remove solids and form a second solution, as shown in Scheme 1.

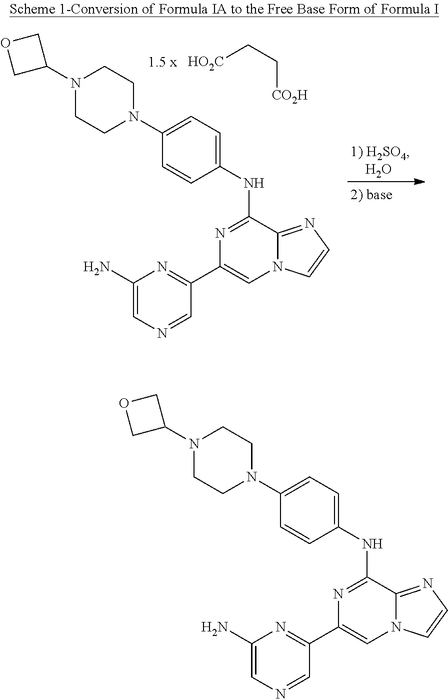 [SMILES: Nc1cncc(-c2cn3ccnc3c(Nc3ccc(N4CCN(C5COC5)CC4)cc3)n2)n1 ; O=C(O)CCC(=O)O ; Nc1cncc(-c2cn3ccnc3c(Nc3ccc(N4CCN(C5COC5)CC4)cc3)n2)n1]

In some embodiments, the base may be selected from sodium carbonate, cesium carbonate, sodium bicarbonate, lithium carbonate, or potassium carbonate. In some embodiments, a solvent, e.g., isopropanol or tetrahydrofuran, is added to the second solution to form a slurry, which is subsequently filtered to isolate Compound I.

In some embodiments, the process further comprises the steps of combining succinic acid with Compound I freebase (i.e., Compound I) to form a crystallization mixture, stirring the crystallization mixture, and isolating the solids in the crystallization mixture. In some embodiments, the isolated solid from the reaction mixture has X-ray powder diffraction pattern that does not exhibit a peak at 19.9 degrees 2θ. The preparation of succinic acid from the free base is generally shown in Scheme 2.

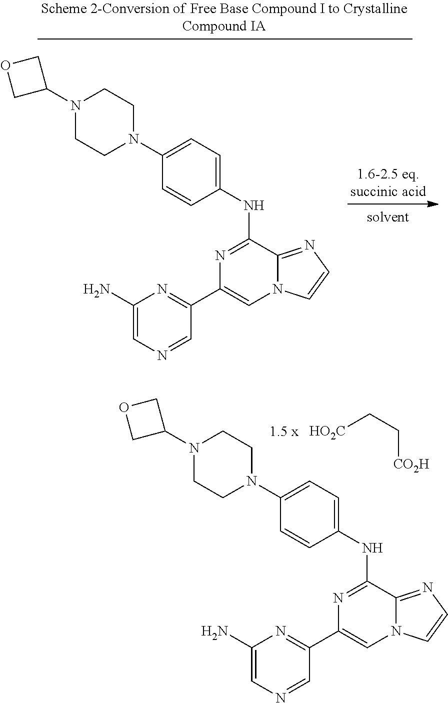

In some embodiments, at least 75% by weight of the solid Compound I isolated by the process described herein is in the form of crystalline Compound of IA.

In some embodiments, at least approximately 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9%, 100%, or within a range approximately defined by any two of the aforementioned values by weight of the solid Compound I isolated by the process described herein is in the form of crystalline Compound IA. For example, at least approximately 75% by weight of the solid Compound I isolated by the process described is in the form of crystalline Compound IA, at least approximately 85% to 99% by weight of the solid Compound I isolated by the process described herein is in the form of crystalline Compound IA, or at least approximately 90% to 99% by weight of the solid Compound I isolated by the process described herein is in the form of crystalline Compound IA.

Crystalline Compound IA can be prepared by dissolving succinic acid in tetrahydrofuran (THF), and then adding the acidic solution to Compound I. Previously described methods using the process used 1.6 molar equivalents of succinic acid. However, utilizing this method to synthesize the crystalline form of Compound IA generated not only crystalline Compound IA, but undesired by-products such as, e.g., Compound I (i.e., the free base).

Without being bound to a particular theory, it was found that control of water content, succinic acid amount, and temperature during the crystallization process resulted in the preparation of pure crystalline Compound IA without the presence of the free base. In particular, it was found to be critical to the synthesis of crystalline Compound IA to have a greater excess of succinic acid. Previous preparations utilized approximately 1.5 molar equivalents of succinic acid to Compound I, and syntheses described herein have utilized more than 1.6 molar equivalents, e.g., 1.7, 1.8, 1.9, or 2.0 equivalents of succinic acid in order to keep the free base (i.e., Compound I) solubilized. The solubility of Compound I is an important factor, as Compound IA has a strong tendency to disproportionate back to a free base form, preventing Compound I free base forms from crystallizing out. FIG. 2 compares the XRPD of the product obtained using the prior processes with that obtained using the process described herein. As demonstrated in FIG. 2, the product obtained using the prior process resulted in a peak at approximately 19.9 degrees 2θ. This peak is believed to be due to the presence of the free base Compound I, which can be seen as a prominent peak in the XRPD for the crystalline free base (FIG. 3). Thus, the previous process produces mixture of Compound I free base and crystalline Compound IA (consisting of 1.5 molecule of succinic acid and 1 molecule of Compound I in the crystal lattice).

In some embodiments, the molar equivalents of succinic acid to Compound I may be approximately 1.6, 1.65, 1.7, 1.75, 1.8, 1.85, 1.9, 1.95, 2.0, 2.05, 2.1, 2.15, 2.2, 2.25, 2.3, 2.35, 2.4, 2.45, 2.5, 2.55, 2.6, 2.65, 2.7, 2.75, 2.8, 2.85, 2.9, 2.95, 3.0, or within a range defined by any two of the aforementioned values. For example, the range of molar equivalents of succinic acid to Compound I may be 1.8 to 2.0, 1.8 to 2.5, 1.6 to 3.0, or 1.6 to 2.5.

In some embodiments, crystalline Compound IA is prepared by combining succinic acid with Compound I to generate the crystalline form, stirring the crystallization mixture, and isolating a solid comprising crystalline Compound IA from the reaction mixture. In these preparations, at least 75% by weight of the product is crystalline Compound IA, wherein crystalline Compound IA exhibits at least three characteristic peaks on X-ray powder diffraction, wherein said characteristic peaks are selected from the group consisting of approximately 8.0, 8.3, 12.1, 16.5, 17.7, 21.8, 23.1, 24.5, 27.1, and 28.4 degrees 2θ. In some embodiments, crystalline Compound IA has an X-ray powder diffraction pattern that does not exhibit a peak at 19.9 degrees 2θ.

In some embodiments, the succinic acid is dissolved in a first solvent before combining the solution with Compound I. In some embodiments, the first solvent used to dissolve succinic acid is chosen from among acetone, isopropyl alcohol, tetrahydrofuran, and 2-methyltetrahydrofuran. In some specific embodiments, the first solvent is tetrahydrofuran.

In some embodiments, wherein the first solvent is tetrahydrofuran, the percent water by weight in the tetrahydrofuran is approximately 1.0% to 10.0%. In some embodiments, wherein the first solvent is tetrahydrofuran, the percent water by weight in the tetrahydrofuran is approximately 1.0% to 5.0%. In some embodiments, wherein the first solvent is tetrahydrofuran, the percent water by weight in the tetrahydrofuran is approximately 1.0% to 3.0%. In some embodiments, wherein the first solvent is tetrahydrofuran, the percent water by weight in the tetrahydrofuran is approximately 1.5% to 2.5%. In some embodiments, wherein the first solvent is tetrahydrofuran, the percent water by weight in the tetrahydrofuran is approximately 1.5% to 2.1%. In some embodiments, wherein the first solvent is tetrahydrofuran, the percent water by weight in the tetrahydrofuran is approximately 1.8%.

In some embodiments, wherein the first solvent is tetrahydrofuran, the percent water content by weight in the tetrahydrofuran may be approximately 1.0%, 1.1%, 1.2%, 1.3%, 1.4%, 1.5%, 1.6%, 1.7%, 1.71%, 1.72%, 1.73%, 1.74%, 1.75%, 1.76%, 1.77%, 1.78%, 1.79%, 1.80%, 1.81%, 1.82%, 1.83%, 1.84%, 1.85%, 1.86%, 1.87%, 1.88%, 1.89%, 1.90%, 1.91%, 1.92%, 1.93%, 1.94%, 1.95%, 1.96%, 1.97%, 1.98%, 1.99%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, 10.0%, or within a range approximately defined by any of the two aforementioned values. For example, in some embodiments, wherein the first solvent is tetrahydrofuran, the percent water by weight in the tetrahydrofuran is approximately 1.5% to 2.1%, or 1.5% to 2.5%, or 1.0% to 5.0%.

In some embodiments, wherein the first solvent is tetrahydrofuran, the percent water by weight in the tetrahydrofuran is approximately 1% to 10%, and the molar ratio of Compound I to succinic acid in the crystallization mixture ranges from a ratio of approximately 1:0.5 to approximately 1:5. In some embodiments, wherein the first solvent is tetrahydrofuran, the percent water by weight in the tetrahydrofuran is approximately 1% to 10%, and the molar ratio of Compound I to succinic acid in the crystallization mixture ranges from a ratio of approximately 1:1 to approximately 1:3. In some embodiments, wherein the first solvent is tetrahydrofuran, the percent water by weight in the tetrahydrofuran is approximately 1% to 10%, and the molar ratio of Compound I to succinic acid in the crystallization mixture ranges from a ratio of approximately 1:1.5 to approximately 1:2.5. In some embodiments, wherein the first solvent is tetrahydrofuran, the percent water by weight in the tetrahydrofuran is approximately 1% to 10%, and the molar ratio of Compound I to succinic acid in the crystallization mixture ranges from a ratio of approximately 1:1.5 to approximately 1:2.1. In some embodiments, wherein the first solvent is tetrahydrofuran, the percent water by weight in the tetrahydrofuran is approximately 1% to 10%, and the molar ratio of Compound I to succinic acid in the crystallization mixture ranges from a ratio of approximately 1:1.5 to approximately 1:2. In some embodiments, wherein the first solvent is tetrahydrofuran, the percent water by weight in the tetrahydrofuran is approximately 1% to 10%, and the molar ratio of Compound I to succinic acid in the crystallization mixture ranges from a ratio of approximately 1:1.5 to approximately 1:1.8. In some embodiments, wherein the first solvent is tetrahydrofuran, the percent water by weight in the tetrahydrofuran is approximately 1% to 10%, and the molar ratio of Compound I to succinic acid in the crystallization mixture is approximately 1:2.1. In some embodiments, wherein the first solvent is tetrahydrofuran, the percent water by weight in the tetrahydrofuran is approximately 1% to 10%, and the molar ratio of Compound I to succinic acid in the crystallization mixture is approximately 1:1.8. In some embodiments, wherein the first solvent is tetrahydrofuran, the percent water by weight in the tetrahydrofuran is approximately 1% to 10%, and the molar ratio of Compound I to succinic acid in the crystallization mixture is approximately 1:2. In some embodiments, wherein the first solvent is tetrahydrofuran, the percent water by weight in the tetrahydrofuran is approximately 1% to 10%, and the molar ratio of Compound I to succinic acid in the crystallization mixture is approximately 1:2.5. In some embodiments, wherein the first solvent is tetrahydrofuran, the percent water by weight in the tetrahydrofuran is approximately 1% to 10%, and the molar ratio of Compound I to succinic acid in the crystallization mixture is approximately 1:1.5. In some embodiments, wherein the first solvent is tetrahydrofuran, the percent water by weight in the tetrahydrofuran is approximately 1% to 10%, and the molar ratio of Compound I to succinic acid in the crystallization mixture is approximately 1:0.5. In some embodiments, wherein the first solvent is tetrahydrofuran, the percent water by weight in the tetrahydrofuran is approximately 1% to 10%, and the molar ratio of Compound I to succinic acid in the crystallization mixture is approximately 1:5.

In some embodiments, wherein the first solvent is tetrahydrofuran, the percent water by weight in the tetrahydrofuran is approximately 1% to 5%, and the molar ratio of Compound I to succinic acid in the crystallization mixture ranges from a ratio of approximately 1:0.5 to approximately 1:5. In some embodiments, wherein the first solvent is tetrahydrofuran, the percent water by weight in the tetrahydrofuran is approximately 1% to 5%, and the molar ratio of Compound I to succinic acid in the crystallization mixture ranges from a ratio of approximately 1:1 to approximately 1:3. In some embodiments, wherein the first solvent is tetrahydrofuran, the percent water by weight in the tetrahydrofuran is approximately 1% to 5%, and the molar ratio of Compound I to succinic acid in the crystallization mixture ranges from a ratio of approximately 1:1.5 to approximately 1:2.5. In some embodiments, wherein the first solvent is tetrahydrofuran, the percent water by weight in the tetrahydrofuran is approximately 1% to 5%, and the molar ratio of Compound I to succinic acid in the crystallization mixture ranges from a ratio of approximately 1:1.5 to approximately 1:2.1. In some embodiments, wherein the first solvent is tetrahydrofuran, the percent water by weight in the tetrahydrofuran is approximately 1% to 5%, and the molar ratio of Compound I to succinic acid in the crystallization mixture ranges from a ratio of approximately 1:1.5 to approximately 1:2. In some embodiments, wherein the first solvent is tetrahydrofuran, the percent water by weight in the tetrahydrofuran is approximately 1% to 5%, and the molar ratio of Compound I to succinic acid in the crystallization mixture ranges from a ratio of approximately 1:1.5 to approximately 1:1.8. In some embodiments, wherein the first solvent is tetrahydrofuran, the percent water by weight in the tetrahydrofuran is approximately 1% to 5%, and the molar ratio of Compound I to succinic acid in the crystallization mixture is approximately 1:2.1. In some embodiments, wherein the first solvent is tetrahydrofuran, the percent water by weight in the tetrahydrofuran is approximately 1% to 5%, and the molar ratio of Compound I to succinic acid in the crystallization mixture is approximately 1:1.8. In some embodiments, wherein the first solvent is tetrahydrofuran, the percent water by weight in the tetrahydrofuran is approximately 1% to 5%, and the molar ratio of Compound I to succinic acid in the crystallization mixture is approximately 1:2. In some embodiments, wherein the first solvent is tetrahydrofuran, the percent water by weight in the tetrahydrofuran is approximately 1% to 5%, and the molar ratio of Compound I to succinic acid in the crystallization mixture is approximately 1:2.5. In some embodiments, wherein the first solvent is tetrahydrofuran, the percent water by weight in the tetrahydrofuran is approximately 1% to 5%, and the molar ratio of Compound I to succinic acid in the crystallization mixture is approximately 1:1.5. In some embodiments, wherein the first solvent is tetrahydrofuran, the percent water by weight in the tetrahydrofuran is approximately 1% to 5%, and the molar ratio of Compound I to succinic acid in the crystallization mixture is approximately 1:0.5. In some embodiments, wherein the first solvent is tetrahydrofuran, the percent water by weight in the tetrahydrofuran is approximately 1% to 5%, and the molar ratio of Compound I to succinic acid in the crystallization mixture is approximately 1:5.

In some embodiments, wherein the first solvent is tetrahydrofuran, the percent water by weight in the tetrahydrofuran is approximately 1% to 3%, and the molar ratio of Compound I to succinic acid in the crystallization mixture ranges from a ratio of approximately 1:0.5 to approximately 1:5. In some embodiments, wherein the first solvent is tetrahydrofuran, the percent water by weight in the tetrahydrofuran is approximately 1% to 3%, and the molar ratio of Compound I to succinic acid in the crystallization mixture ranges from a ratio of approximately 1:1 to approximately 1:3. In some embodiments, wherein the first solvent is tetrahydrofuran, the percent water by weight in the tetrahydrofuran is approximately 1% to 3%, and the molar ratio of Compound I to succinic acid in the crystallization mixture ranges from a ratio of approximately 1:1.5 to approximately 1:2.5. In some embodiments, wherein the first solvent is tetrahydrofuran, the percent water by weight in the tetrahydrofuran is approximately 1% to 3%, and the molar ratio of Compound I to succinic acid in the crystallization mixture ranges from a ratio of approximately 1:1.5 to approximately 1:2.1. In some embodiments, wherein the first solvent is tetrahydrofuran, the percent water by weight in the tetrahydrofuran is approximately 1% to 3%, and the molar ratio of Compound I to succinic acid in the crystallization mixture ranges from a ratio of approximately 1:1.5 to approximately 1:2. In some embodiments, wherein the first solvent is tetrahydrofuran, the percent water by weight in the tetrahydrofuran is approximately 1% to 3%, and the molar ratio of Compound I to succinic acid in the crystallization mixture ranges from a ratio of approximately 1:1.5 to approximately 1:1.8. In some embodiments, wherein the first solvent is tetrahydrofuran, the percent water by weight in the tetrahydrofuran is approximately 1% to 3%, and the molar ratio of Compound I to succinic acid in the crystallization mixture is approximately 1:2.1. In some embodiments, wherein the first solvent is tetrahydrofuran, the percent water by weight in the tetrahydrofuran is approximately 1% to 3%, and the molar ratio of Compound I to succinic acid in the crystallization mixture is approximately 1:1.8. In some embodiments, wherein the first solvent is tetrahydrofuran, the percent water by weight in the tetrahydrofuran is approximately 1% to 3%, and the molar ratio of Compound I to succinic acid in the crystallization mixture is approximately 1:2. In some embodiments, wherein the first solvent is tetrahydrofuran, the percent water by weight in the tetrahydrofuran is approximately 1% to 3%, and the molar ratio of Compound I to succinic acid in the crystallization mixture is approximately 1:2.5. In some embodiments, wherein the first solvent is tetrahydrofuran, the percent water by weight in the tetrahydrofuran is approximately 1% to 3%, and the molar ratio of Compound I to succinic acid in the crystallization mixture is approximately 1:1.5. In some embodiments, wherein the first solvent is tetrahydrofuran, the percent water by weight in the tetrahydrofuran is approximately 1% to 3%, and the molar ratio of Compound I to succinic acid in the crystallization mixture is approximately 1:0.5. In some embodiments, wherein the first solvent is tetrahydrofuran, the percent water by weight in the tetrahydrofuran is approximately 1% to 3%, and the molar ratio of Compound I to succinic acid in the crystallization mixture is approximately 1:5.

In some embodiments, wherein the first solvent is tetrahydrofuran, the percent water by weight in the tetrahydrofuran is approximately 1.5% to 2.5%, and the molar ratio of Compound I to succinic acid in the crystallization mixture ranges from a ratio of approximately 1:0.5 to approximately 1:5. In some embodiments, wherein the first solvent is tetrahydrofuran, the percent water by weight in the tetrahydrofuran is approximately 1.5% to 2.5%, and the molar ratio of Compound I to succinic acid in the crystallization mixture ranges from a ratio of approximately 1:1 to approximately 1:3. In some embodiments, wherein the first solvent is tetrahydrofuran, the percent water by weight in the tetrahydrofuran is approximately 1.5% to 2.5%, and the molar ratio of Compound I to succinic acid in the crystallization mixture ranges from a ratio of approximately 1:1.5 to approximately 1:2.5. In some embodiments, wherein the first solvent is tetrahydrofuran, the percent water by weight in the tetrahydrofuran is approximately 1.5% to 2.5%, and the molar ratio of Compound I to succinic acid in the crystallization mixture ranges from a ratio of approximately 1:1.5 to approximately 1:2.1. In some embodiments, wherein the first solvent is tetrahydrofuran, the percent water by weight in the tetrahydrofuran is approximately 1.5% to 2.5%, and the molar ratio of Compound I to succinic acid in the crystallization mixture ranges from a ratio of approximately 1:1.5 to approximately 1:2. In some embodiments, wherein the first solvent is tetrahydrofuran, the percent water by weight in the tetrahydrofuran is approximately 1.5% to 2.5%, and the molar ratio of Compound I to succinic acid in the crystallization mixture ranges from a ratio of approximately 1:1.5 to approximately 1:1.8. In some embodiments, wherein the first solvent is tetrahydrofuran, the percent water by weight in the tetrahydrofuran is approximately 1.5% to 2.5%, and the molar ratio of Compound I to succinic acid in the crystallization mixture is approximately 1:2.1. In some embodiments, wherein the first solvent is tetrahydrofuran, the percent water by weight in the tetrahydrofuran is approximately 1.5% to 2.5%, and the molar ratio of Compound I to succinic acid in the crystallization mixture is approximately 1:1.8. In some embodiments, wherein the first solvent is tetrahydrofuran, the percent water by weight in the tetrahydrofuran is approximately 1.5% to 2.5%, and the molar ratio of Compound I to succinic acid in the crystallization mixture is approximately 1:2. In some embodiments, wherein the first solvent is tetrahydrofuran, the percent water by weight in the tetrahydrofuran is approximately 1.5% to 2.5%, and the molar ratio of Compound I to succinic acid in the crystallization mixture is approximately 1:2.5. In some embodiments, wherein the first solvent is tetrahydrofuran, the percent water by weight in the tetrahydrofuran is approximately 1.5% to 2.5%, and the molar ratio of Compound I to succinic acid in the crystallization mixture is approximately 1:1.5. In some embodiments, wherein the first solvent is tetrahydrofuran, the percent water by weight in the tetrahydrofuran is approximately 1.5% to 2.5%, and the molar ratio of Compound I to succinic acid in the crystallization mixture is approximately 1:0.5. In some embodiments, wherein the first solvent is tetrahydrofuran, the percent water by weight in the tetrahydrofuran is approximately 1.5% to 2.5%, and the molar ratio of Compound I to succinic acid in the crystallization mixture is approximately 1:5.

In some embodiments, wherein the first solvent is tetrahydrofuran, the percent water by weight in the tetrahydrofuran is approximately 1.5% to 2.1%, and the molar ratio of Compound I to succinic acid in the crystallization mixture ranges from a ratio of approximately 1:0.5 to approximately 1:5. In some embodiments, wherein the first solvent is tetrahydrofuran, the percent water by weight in the tetrahydrofuran is approximately 1.5% to 2.1%, and the molar ratio of Compound I to succinic acid in the crystallization mixture ranges from a ratio of approximately 1:1 to approximately 1:3. In some embodiments, wherein the first solvent is tetrahydrofuran, the percent water by weight in the tetrahydrofuran is approximately 1.5% to 2.1%, and the molar ratio of Compound I to succinic acid in the crystallization mixture ranges from a ratio of approximately 1:1.5 to approximately 1:2.5. In some embodiments, wherein the first solvent is tetrahydrofuran, the percent water by weight in the tetrahydrofuran is approximately 1.5% to 2.1%, and the molar ratio of Compound I to succinic acid in the crystallization mixture ranges from a ratio of approximately 1:1.5 to approximately 1:2.1. In some embodiments, wherein the first solvent is tetrahydrofuran, the percent water by weight in the tetrahydrofuran is approximately 1.5% to 2.1%, and the molar ratio of Compound I to succinic acid in the crystallization mixture ranges from a ratio of approximately 1:1.5 to approximately 1:2. In some embodiments, wherein the first solvent is tetrahydrofuran, the percent water by weight in the tetrahydrofuran is approximately 1.5% to 2.1%, and the molar ratio of Compound I to succinic acid in the crystallization mixture ranges from a ratio of approximately 1:1.5 to approximately 1:1.8. In some embodiments, wherein the first solvent is tetrahydrofuran, the percent water by weight in the tetrahydrofuran is approximately 1.5% to 2.1%, and the molar ratio of Compound I to succinic acid in the crystallization mixture is approximately 1:2.1. In some embodiments, wherein the first solvent is tetrahydrofuran, the percent water by weight in the tetrahydrofuran is approximately 1.5% to 2.1%, and the molar ratio of Compound I to succinic acid in the crystallization mixture is approximately 1:1.8. In some embodiments, wherein the first solvent is tetrahydrofuran, the percent water by weight in the tetrahydrofuran is approximately 1.5% to 2.1%, and the molar ratio of Compound I to succinic acid in the crystallization mixture is approximately 1:2. In some embodiments, wherein the first solvent is tetrahydrofuran, the percent water by weight in the tetrahydrofuran is approximately 1.5% to 2.1%, and the molar ratio of Compound I to succinic acid in the crystallization mixture is approximately 1:2.5. In some embodiments, wherein the first solvent is tetrahydrofuran, the percent water by weight in the tetrahydrofuran is approximately 1.5% to 2.1%, and the molar ratio of Compound I to succinic acid in the crystallization mixture is approximately 1:1.5. In some embodiments, wherein the first solvent is tetrahydrofuran, the percent water by weight in the tetrahydrofuran is approximately 1.5% to 2.1%, and the molar ratio of Compound I to succinic acid in the crystallization mixture is approximately 1:0.5. In some embodiments, wherein the first solvent is tetrahydrofuran, the percent water by weight in the tetrahydrofuran is approximately 1.5% to 2.1%, and the molar ratio of Compound I to succinic acid in the crystallization mixture is approximately 1:5.

In some embodiments, wherein the first solvent is tetrahydrofuran, the percent water by weight in the tetrahydrofuran is approximately 1.8%, and the molar ratio of Compound I to succinic acid in the crystallization mixture ranges from a ratio of approximately 1:0.5 to approximately 1:5. In some embodiments, wherein the first solvent is tetrahydrofuran, the percent water by weight in the tetrahydrofuran is approximately 1.8%, and the molar ratio of Compound I to succinic acid in the crystallization mixture ranges from a ratio of approximately 1:1 to approximately 1:3. In some embodiments, wherein the first solvent is tetrahydrofuran, the percent water by weight in the tetrahydrofuran is approximately 1.8%, and the molar ratio of Compound I to succinic acid in the crystallization mixture ranges from a ratio of approximately 1:1.5 to approximately 1:2.5. In some embodiments, wherein the first solvent is tetrahydrofuran, the percent water by weight in the tetrahydrofuran is approximately 1.8%, and the molar ratio of Compound I to succinic acid in the crystallization mixture ranges from a ratio of approximately 1:1.5 to approximately 1:2.1. In some embodiments, wherein the first solvent is tetrahydrofuran, the percent water by weight in the tetrahydrofuran is approximately 1.8%, and the molar ratio of Compound I to succinic acid in the crystallization mixture ranges from a ratio of approximately 1:1.5 to approximately 1:2. In some embodiments, wherein the first solvent is tetrahydrofuran, the percent water by weight in the tetrahydrofuran is approximately 1.8%, and the molar ratio of Compound I to succinic acid in the crystallization mixture ranges from a ratio of approximately 1:1.5 to approximately 1:1.8. In some embodiments, wherein the first solvent is tetrahydrofuran, the percent water by weight in the tetrahydrofuran is approximately 1.8%, and the molar ratio of Compound I to succinic acid in the crystallization mixture is approximately 1:2.1. In some embodiments, wherein the first solvent is tetrahydrofuran, the percent water by weight in the tetrahydrofuran is approximately 1.8%, and the molar ratio of Compound I to succinic acid in the crystallization mixture is approximately 1:1.8. In some embodiments, wherein the first solvent is tetrahydrofuran, the percent water by weight in the tetrahydrofuran is approximately 1.8%, and the molar ratio of Compound I to succinic acid in the crystallization mixture is approximately 1:2. In some embodiments, wherein the first solvent is tetrahydrofuran, the percent water by weight in the tetrahydrofuran is approximately 1.8%, and the molar ratio of Compound I to succinic acid in the crystallization mixture is approximately 1:2.5. In some embodiments, wherein the first solvent is tetrahydrofuran, the percent water by weight in the tetrahydrofuran is approximately 1.8%, and the molar ratio of Compound I to succinic acid in the crystallization mixture is approximately 1:1.5. In some embodiments, wherein the first solvent is tetrahydrofuran, the percent water by weight in the tetrahydrofuran is approximately 1.8%, and the molar ratio of Compound I to succinic acid in the crystallization mixture is approximately 1:0.5. In some embodiments, wherein the first solvent is tetrahydrofuran, the percent water by weight in the tetrahydrofuran is approximately 1.8%, and the molar ratio of Compound I to succinic acid in the crystallization mixture is approximately 1:5.

In some embodiments, the molar ratio of the Compound I to succinic acid in the crystallization mixture ranges from a ratio of approximately 1:0.5 to approximately 1:5. In some embodiments, the molar ratio of Compound I to succinic acid in the crystallization mixture ranges from a ratio of approximately 1:1 to approximately 1:3. In some embodiments, the molar ratio of Compound I to succinic acid in the crystallization mixture ranges from a ratio of approximately 1:1.5 to approximately 1:2.5. In some embodiments, the molar ratio of Compound I to succinic acid in the crystallization mixture ranges from a ratio of approximately 1:1.5 to approximately 1:2.1. In some embodiments, the molar ratio of Compound I to succinic acid in the crystallization mixture ranges from a ratio of approximately 1:1.5 to approximately 1:2. In some embodiments, the molar ratio of Compound I to succinic acid in the crystallization mixture ranges from a ratio of approximately 1:1.5 to approximately 1:1.8. In some embodiments, the molar ratio of Compound I to succinic acid in the crystallization mixture is approximately 1:2.1. In some embodiments, the molar ratio of Compound I to succinic acid in the crystallization mixture is approximately 1:1.8. In some embodiments, the molar ratio of Compound I to succinic acid in the crystallization mixture is approximately 1:2. In some embodiments, the molar ratio of Compound I to succinic acid in the crystallization mixture is approximately 1:2.5. In some embodiments, the molar ratio of Compound I to succinic acid in the crystallization mixture is approximately 1:1.5. In some embodiments, the molar ratio of Compound I to succinic acid in the crystallization mixture is approximately 1:0.5. In some embodiments, the molar ratio of Compound I to succinic acid in the crystallization mixture is approximately 1:5.

In some embodiments, wherein the first solvent is tetrahydrofuran, the percent water content by weight in the tetrahydrofuran may be approximately 1.0%, 1.1%, 1.2%, 1.3%, 1.4%, 1.5%, 1.6%, 1.7%, 1.71%, 1.72%, 1.73%, 1.74%, 1.75%, 1.76%, 1.77%, 1.78%, 1.79%, 1.80%, 1.81%, 1.82%, 1.83%, 1.84%, 1.85%, 1.86%, 1.87%, 1.88%, 1.89%, 1.90%, 1.91%, 1.92%, 1.93%, 1.94%, 1.95%, 1.96%, 1.97%, 1.98%, 1.99%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, 10.0%, or within a range approximately defined by any of the two aforementioned values, and the molar ratio of crystalline Compound I to succinic acid may be approximately 1:0.5, 1:1, 1:1.1, 1:1.2, 1:1.3, 1:1.4, 1:1.5, 1:1.6, 1:1.7, 1:1.8, 1:1.9, 1:2, 1:2.5, 1:3, 1:3.5, 1:4, 1:4.5, 1:5, or within a range approximately defined by any of the two aforementioned values. For example, in some embodiments, wherein the first solvent is tetrahydrofuran, the percent water by weight in the tetrahydrofuran is approximately 1.5% to 2.1% and the molar ratio of crystalline Compound I to succinic acid is approximately 1:1.8, or 1.5% to 2.5% and the molar ratio of crystalline Compound I to succinic acid is approximately 1:0.5 to 1:1.8, or 1.0% to 5.0% and the molar ratio of crystalline Compound I to succinic acid is approximately 1:0.5 to 1:5.

In some embodiments, the second solution may be heated at approximately 35° C., 35.5° C., 36° C., 36.5° C., 37° C., 37.5° C., 38° C., 38.5° C., 39° C., 39.5° C., 40° C., 40.5° C., 41° C., 41.5° C., 42° C., 42.5° C., 43° C., 43.5° C., 44° C., 44.5° C., 45° C., or within a range approximately defined by any of the two aforementioned values. For example, the second solution may be heated between approximately 37° C. to 42° C., or approximately 40° C.

In some embodiments, the second solution is added to the first solution in order to form the crystallization mixture. In some embodiments, the first solution is added to the second solution in order to form the crystallization mixture. In some embodiments, the first solution is added to the second solution in order to form the crystallization mixture over a period of approximately 10 minutes to 120 minutes. In some embodiments, the first solution is added to the second solution in order to form the crystallization mixture over a period of approximately 20 minutes to 60 minutes. In some embodiments, the first solution is added to the second solution in order to form the crystallization mixture over a period of approximately 20 minutes to 30 minutes. In some embodiments, the first solution is added to the second solution in order to form the crystallization mixture over a period of approximately 30 minutes.

In some embodiments, the first solution may be added to the second solution over a period of approximately 10 minutes, 15 minutes, 20 minutes, 25 minutes, 30 minutes, 35 minutes, 40 minutes, 45 minutes, 50 minutes, 55 minutes, 60 minutes, 65 minutes, 70 minutes, 75 minutes, 80 minutes, 85 minutes, 90 minutes, 95 minutes, 100 minutes, 105 minutes, 110 minutes, 115 minutes, 120 minutes, or within a range approximately defined by any of the two aforementioned values. For example, the first solution may be added to the second solution over a period of approximately 10 minutes to 120 minutes, 30 minutes, or 20 minutes to 60 minutes.

In some embodiments, the crystallization mixture may be stirred at approximately 35° C. to 45° C. In some embodiments, the crystallization mixture may be stirred at approximately 37° C. to 42° C. In some embodiments, the crystallization mixture may be stirred at approximately 40° C.

In some embodiments, the crystallization mixture may be stirred at approximately 35° C., 35.5° C., 36° C., 36.5° C., 37° C., 37.5° C., 38° C., 38.5° C., 39° C., 39.5° C., 40° C., 40.5° C., 41° C., 41.5° C., 42° C., 42.5° C., 43° C., 43.5° C., 44° C., 44.5° C., 45° C., or within a range approximately defined by any of the two aforementioned values. For example, the crystallization mixture may be stirred between approximately 37° C. to 42° C., or approximately 40° C.

In some embodiments, the crystallization mixture is stirred approximately between 8 hours to 36 hours. In some embodiments, the crystallization mixture is stirred approximately between 12 hours to 24 hours. In some embodiments, the crystallization mixture is stirred approximately between 15 hours to 21 hours. In some embodiments, the crystallization mixture is stirred approximately 18 hours.

In some embodiments, the crystallization mixture is stirred approximately between 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, or within a range approximately defined by any of the two aforementioned values. For example, the crystallization mixture is stirred approximately between 8 hours and 24 hours, 12 hours and 24 hours, or approximately 24 hours.

To prevent carryover of mixed solid forms as impurities, due to disproportionation during manufacturing of crystalline Compound IA, into the formulation and granulation of crystalline Compound IA oral dosage tablet, and to ensure consistent product performance on use and storage, the invention describes a quality control test method to ensure that crystalline compound IA cocrystal compositionally pure.

In some embodiments, crystalline Compound IA may be removed from the crystallization mixture and analyzed for total succinic acid content. In some embodiments, the crystallization mixture may be stirred for approximately 2 hours to 6 hours the crystalline Compound IA contains less than approximately 27% succinate or succinic acid by weight.

In some embodiments, crystalline Compound IA may contain approximately 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35% by weight succinate or succinic acid, or a range approximately defined by any of the two aforementioned values. For example, crystalline Compound IA may contain approximately 20% by weight succinate or succinic acid, 27% by weight succinic acid or succinate, or between 20% and 27% by weight succinic acid or succinate.

In some embodiments, Compound IA in the crystallization reaction is a crystal form exhibiting an X-ray powder diffraction pattern comprising at least three characteristic peak, wherein said characteristic peak is selected from the group consisting of approximately 8.0, 8.3, 12.1, 16.5, 17.7, 21.8, 24.5, and 28.4 degrees 2θ. In some embodiments, Compound IA in the crystallization reaction is a crystal form exhibiting an X-ray powder diffraction pattern comprising at least three characteristic peak, wherein said characteristic peak is selected from the group consisting of approximately 8.0, 8.3, 12.1, 16.5, 17.7, 21.8, 24.5, and 28.4 degrees 2θ. In some embodiments, Compound IA in the crystallization reaction is a crystal form exhibiting an X-ray powder diffraction pattern comprising at least three characteristic peak, wherein said characteristic peak is selected from the group consisting of approximately 8.0, 8.3, 12.1, 16.5, 17.7, 21.8, 24.5, and 28.4 degrees 2θ.

In some embodiments, the succinic acid content of the product generated from the crystallization reaction to form crystalline Compound IA is below approximately 27% by weight. In such embodiments, the succinic acid content of the product generated from the crystallization reaction to form crystalline Compound IA is below approximately 27% by weight and the crystallization reaction is stirred for additional time, e.g., an addition 1, 2, 3, 4, 5, 6, 7, 8, 9, 10, 11, or 12 hours, or more.

In some embodiments, the isolated solid from the crystallization reaction comprises greater than approximately 75% by weight crystalline Compound IA. In some embodiments, the isolated solid from the crystallization reaction comprises greater than approximately 80% by weight crystalline Compound IA. In some embodiments, the isolated solid from the crystallization reaction comprises greater than approximately 85% by weight crystalline Compound IA. In some embodiments, the isolated solid from the crystallization reaction comprises greater than approximately 90% by weight crystalline Compound IA. In some embodiments, the isolated solid from the crystallization reaction comprises greater than approximately 95% by weight crystalline Compound IA. In some embodiments, the isolated solid from the crystallization reaction comprises greater than approximately 98% by weight crystalline Compound IA, for example greater than 98.1%, 98.2%, 98.3%, 98.4%, 98.5%, 98.6%, 98.7%, 98.8%, or 98.9% by weight. In some embodiments, the isolated solid from the crystallization reaction comprises greater than approximately 99% by weight crystalline Compound IA, for example greater than 99.1%, 99.2%, 99.3%, 99.4%, 99.5%, 99.6%, 99.7%, 99.8%, or 99.9% by weight. In some embodiments, the isolated solid from the crystallization reaction comprises greater than approximately 99.9% by weight crystalline Compound IA. In some embodiments, the isolated solid from the crystallization reaction comprises greater than approximately 85% by weight to 99.9% by weight crystalline Compound IA. In some embodiments, the isolated solid from the crystallization reaction comprises greater than approximately 85% by weight to approximately 99% by weight crystalline Compound IA. In some embodiments, the isolated solid from the crystallization reaction comprises greater than approximately 90% by weight to 99.9% by weight crystalline Compound IA. In some embodiments, the isolated solid from the crystallization reaction comprises greater than approximately 90% by weight to approximately 99% by weight crystalline Compound IA. In some embodiments, the isolated solid from the crystallization reaction comprises greater than approximately 95% by weight to 99.9% by weight crystalline Compound IA. In some embodiments, the isolated solid from the crystallization reaction comprises greater than approximately 95% by weight to approximately 99% by weight crystalline Compound IA. In some embodiments, the isolated solid from the crystallization reaction comprises greater than approximately 98% by weight to approximately 99.9% by weight crystalline Compound IA. In some embodiments, the isolated solid from the crystallization reaction comprises greater than approximately 98% by weight to 99.9% by weight crystalline Compound IA. In some embodiments, the isolated solid from the crystallization reaction comprises greater than approximately 99% by weight to 99.9% by weight crystalline Compound IA.

In some embodiments, the molar ratio of Compound I to succinic acid in the crystallization mixture ranges from a ratio of approximately 1:0.5 to approximately 1:5. In some embodiments, the molar ratio of Compound I to succinic acid in the crystallization mixture ranges from a ratio of approximately 1:1 to approximately 1:3. In some embodiments, the molar ratio of Compound I to succinic acid in the crystallization mixture ranges from a ratio of approximately 1:1.5 to approximately 1:2.5. In some embodiments, the molar ratio of Compound I to succinic acid in the crystallization mixture ranges from a ratio of approximately 1:1.5 to approximately 1:2.1. In some embodiments, the molar ratio of Compound I to succinic acid in the crystallization mixture ranges from a ratio of approximately 1:1.5 to approximately 1:2. In some embodiments, the molar ratio of Compound I to succinic acid in the crystallization mixture ranges from a ratio of approximately 1:1.5 to approximately 1:1.8. In some embodiments, the molar ratio of Compound I to succinic acid in the crystallization mixture is approximately 1:2.1. In some embodiments, the molar ratio of Compound I to succinic acid in the crystallization mixture is approximately 1:1.8. In some embodiments, the molar ratio of Compound I to succinic acid in the crystallization mixture is approximately 1:2. In some embodiments, the molar ratio of Compound I to succinic acid in the crystallization mixture is approximately 1:2.5. In some embodiments, the molar ratio of Compound I to succinic acid in the crystallization mixture is approximately 1:1.5. In some embodiments, the molar ratio of Compound I to succinic acid in the crystallization mixture is approximately 1:0.5. In some embodiments, the molar ratio of Compound I to succinic acid in the crystallization mixture is approximately 1:5.

In some embodiments, the process to generate the crystalline Compound IA may further comprise a step to recover wet Compound IA through filtration of the crystallization mixture. In some embodiments, the wet compound of Formula I may be dried at approximately 50° C. to approximately 70° C., for example at 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., or 70° C. In some embodiments, an X-ray powder diffraction pattern of the isolated solid crystalline Compound IA does not exhibit a peak at approximately 19.9 degrees 2θ.

### Pharmaceutical Compositions

Compound IA (i.e., Compound I sesquisuccinate) and any polymorphic forms thereof described herein, can be administered as the neat chemical, but it is typical, to administer the compound, or polymorph thereof, in the form of a pharmaceutical composition or formulation. Provided are pharmaceutical compositions comprising: (i) crystalline Compound I provided herein (ii) a pharmaceutical carrier, excipient, adjuvant, or vehicle. Pharmaceutical carrier, excipient, adjuvant, or vehicle may also be referred to herein as pharmaceutically acceptable carrier, excipient, adjuvant or vehicle or as biocompatible pharmaceutical carrier, excipient, adjuvant, or vehicle. The composition can include a crystalline Compound I provided herein either as the sole active agent or in combination with other agents, such as oligo- or polynucleotides, oligo- or polypeptides, drugs, or hormones mixed with one or more pharmaceutically acceptable carriers or excipients. Carriers, excipients, and other ingredients can be deemed pharmaceutically acceptable insofar as they are compatible with other ingredients of the formulation and not deleterious to the recipient thereof.

The term “carrier” refers to diluents, disintegrants, precipitation inhibitors, surfactants, glidants, binders, lubricants, and other excipients and vehicles with which the compound is administered. Carriers are generally described herein and also in “Remington's Pharmaceutical Sciences” by E. W. Martin.

The pharmaceutical compositions can be formulated to contain suitable pharmaceutically acceptable carriers, including excipients and auxiliaries that facilitate processing of the polymorphic forms described herein into preparations that can be used pharmaceutically. The mode of administration generally determines the nature of the carrier. For example, formulations for parenteral administration can include aqueous solutions of the active compounds in water-soluble form. Carriers suitable for parenteral administration can be selected from among saline, buffered saline, dextrose, water, and other physiologically compatible solutions. Exemplary carriers for parenteral administration are physiologically compatible buffers such as Hanks's solution, Ringer's solution, or physiologically buffered saline. For tissue or cellular administration, penetrants appropriate to the particular barrier to be permeated are used in the formulation. Such penetrants are generally known in the art. For preparations including proteins, the formulation can include stabilizing materials, such as polyols (such as, sucrose) and/or surfactants (such as, nonionic surfactants), and the like.

Alternatively, formulations for parenteral use can include dispersions or suspensions of polymorphic forms described herein prepared as appropriate oily injection suspensions. Suitable lipophilic solvents or vehicles include fatty oils, such as sesame oil, and synthetic fatty acid esters, such as ethyl oleate or triglycerides, or liposomes. Aqueous injection suspensions can contain substances that increase the viscosity of the suspension, such as sodium carboxymethylcellulose, sorbitol, dextran, and mixtures thereof. Optionally, the suspension also can contain suitable stabilizers or agents that increase the solubility of the compounds to allow for the preparation of highly concentrated solutions. Aqueous polymers that provide pH-sensitive solubilization and/or sustained release of the active agent also can be used as coatings or matrix structures, such as methacrylic polymers, such as the EUDRAGIT™ series available from Rohm America Inc. (Piscataway, N.J.). Emulsions, such as, oil-in-water and water-in-oil dispersions, also can be used, optionally stabilized by an emulsifying agent or dispersant (surface active materials; surfactants). Suspensions can contain suspending agents such as ethoxylated isostearyl alcohols, polyoxyethlyene sorbitol and sorbitan esters, microcrystalline cellulose, aluminum metahydroxide, bentonite, agar-agar, gum tragacanth, and mixtures thereof.

Liposomes containing the polymorphic forms described herein also can be employed for parenteral administration. Liposomes generally are derived from phospholipids or other lipid substances. The compositions in liposome form also can contain other ingredients, such as stabilizers, preservatives, excipients, and the like. Exemplary lipids include phospholipids and phosphatidyl cholines (lecithins), both natural and synthetic. Methods of forming liposomes are known in the art. See, e.g., Prescott (Ed.), Methods in Cell Biology, Vol. XIV, p. 33, Academic Press, New York (1976).

In some embodiments, the polymorph, or composition thereof, disclosed herein is formulated for oral administration using pharmaceutically acceptable carriers well known in the art. Preparations formulated for oral administration can be in the form of tablets, pills, capsules, cachets, dragees, lozenges, liquids, gels, syrups, slurries, elixirs, suspensions, or powders. To illustrate, pharmaceutical preparations for oral use can be obtained by combining the active compounds with a solid excipient, optionally grinding the resulting mixture, and processing the mixture of granules, after adding suitable auxiliaries if desired, to obtain tablets or dragee cores. Oral formulations can employ liquid carriers similar in type to those described for parenteral use, such as, buffered aqueous solutions, suspensions, and the like.

Exemplary oral formulations include tablets, dragees, and gelatin capsules. These preparations can contain one or more excipients, which include, without limitation: a) diluents, such as microcrystalline cellulose and sugars, including lactose, dextrose, sucrose, mannitol, or sorbitol; b) binders, such as sodium starch glycolate, croscarmellose sodium, magnesium aluminum silicate, starch from corn, wheat, rice, potato, etc.; c) cellulose materials, such as methylcellulose, hydroxypropylmethyl cellulose, and sodium carboxymethylcellulose, polyvinylpyrrolidone, gums, such as gum arabic and gum tragacanth, and proteins, such as gelatin and collagen; d) disintegrating or solubilizing agents such as cross-linked polyvinyl pyrrolidone, starches, agar, alginic acid or a salt thereof, such as sodium alginate, or effervescent compositions; e) lubricants, such as silica, talc, stearic acid or its magnesium or calcium salt, and polyethylene glycol; f) flavorants and sweeteners; g) colorants or pigments, such as, to identify the product or to characterize the quantity (dosage) of active compound; and h) other ingredients, such as preservatives, stabilizers, swelling agents, emulsifying agents, solution promoters, salts for regulating osmotic pressure, and buffers.

Examples of carriers include, but are not limited to, aluminum monostearate, aluminum stearate, carboxymethylcellulose, carboxymethylcellulose sodium, crospovidone, glyceryl isostearate, glyceryl monostearate, hydroxyethylcellulose, hydroxymethylcellulose, hydroxyoctacosanyl hydroxystearate, hydroxypropylcellulose, hydroxypropylmethylcellulose, lactose, lactose monohydrate, magnesium stearate, mannitol, microcrystalline cellulose, poloxamer 124, poloxamer 181, poloxamer 182, poloxamer 188, poloxamer 237, poloxamer 407, povidone, silicon dioxide, colloidal silicon dioxide, silicone, silicone adhesive 4102, and silicone emulsion. It should be understood, however, that the carriers selected for the pharmaceutical compositions provided in the present disclosure, and the amounts of such carriers in the composition, may vary depending on the method of formulation (such as, dry granulation formulation, solid dispersion formulation).

In certain variations, the pharmaceutical composition comprises crystalline Compound I provided herein and at least one pharmaceutically acceptable carrier selected from the group consisting of hydroxypropylmethylcellulose, mannitol, crospovidone, poloxamer, colloidal silicon dioxide, microcrystalline cellulose, magnesium stearate, and any mixtures thereof. In another variation, the pharmaceutical composition comprises crystalline Compound I provided herein, and at least one additionally pharmaceutically acceptable carrier selected from the group consisting of mannitol, crospovidone, poloxamer, colloidal silicon dioxide, microcrystalline cellulose, magnesium stearate, and any mixtures thereof.

It should also be understood that the pharmaceutically acceptable carriers described above may perform one or more different functions in a given formulation, and may fall within one or more functional classes of carriers (such as, disintegrants, lubricants, diluents).

It should further be understood that, in other embodiments, the pharmaceutical composition may comprise one or more additional carriers to improve flow, compression, hardness, taste, and tablet performance.

### EXAMPLES

### Example 1A: Method of Converting Compound IA into Compound I

Bulk Compound I sesquisuccinate (Compound IA) containing residual free base was initially reconverted into Compound I (i.e., the free base form), as shown in Scheme 1. First, crude crystalline Compound I sesquisuccinate (330 g, 0.5 moles) was treated with sulfuric acid (46.9 g, 0.48 moles) to obtain the water soluble sulfate of Compound I, which allowed for polish filtration. Subsequently, Compound I sulfate was converted into the free base via basification with sodium carbonate (197 g, 1.86 moles). The free base precipitated and subsequently slurried into a water-isopropanol solution at 60° C. for two hours before filtration to isolate the material. The free base form was used as an amorphous solid to prepare pure crystalline Compound IA.

### Example 1B: Method of Converting Compound IA into Crystalline Compound IA

Free base Compound I (225 g, 0.5 moles) was first converted into crystalline Compound IA in a solid-to-solid transformation in 4 parts of tetrahydrofuran and heating at 40° C., before a solution of succinic acid (1.8 equivalents: 107.8 g, 0.9 moles) in 9 parts tetrahydrofuran was added. The solution was then thoroughly mixed, and the resulting suspension thoroughly stirred for not less than 18 hours at 40° C. to avoid precipitation of the free base. The mixture was then cooled, filtered, and the pure crystalline Compound IA was washed with tetrahydrofuran and dried at no more than 60° C. under vacuum.

### Example 2: Preparation Conditions for Converting Compound I into Crystalline Compound IA

Crystalline Compound IA was prepared from various crystalline forms of Compound I and amorphous Compound I with various amounts of succinic acid (>1.8 equivalents). Compound I was added into tetrahydrofuran (13 equivalents) at approximately 40° C., followed by addition of succinic acid (1.8-2.5 equivalents). The mixture was thoroughly mixed for approximately 18 hours prior to the isolation and drying of the product.

Table 2 summarizes the preparation conditions for crystalline Compound IA using either the crystalline forms of Compound I or amorphous form of Compound I, and various equivalents of succinic acid. Additionally, water content in the tetrahydrofuran each preparation was measured and adjusted with respect to the amount of Compound I. It was unexpectedly found that high level of water in the tetrahydrofuran (10% by weight) led to disproportionation of crystalline Compound IA. The succinic acid equivalents relative to the Compound I are greater than 1.6 molar equivalents and the percent water content by weight relative to Compound I. The preparations were mixed thoroughly to prevent local wet spot and disproportionation.

As described in Table 2 below, an effective ratio of succinic acid to Compound I was discovered to be approximately 1.8, and percent water content by weight within the tetrahydrofuran was effectively determined to be approximately 1.8% by weight to generate a desirable composition of crystalline Compound IA.

TABLE 2
Summary of preparation conditions for crystalline Compound IA

| | | Percent | |
|---|---|---|---|
| | Molar | water | |
| | equivalents | content by | |
| | of succinic | weight in | |
| | acid | tetrahydrofuran | |
| Form of | relative to | relative to | |
| Compound I | Compound I | Compound I | Product |
| Crystalline | 1.8 | 1.84% | Crystalline Compound IA |
| Form XVI | | | with XRPD peaks at |
| | | | approximately 8.0, 8.3, |
| | | | 12.1, 16.5, 17.7, 21.8, |
| | | | 23.1, 24.5, 27.1, and |
| | | | 28.4 degrees 2θ. |
| Crystalline | 2.0 | 1.84% | Crystalline Compound IA |
| Form XVI | | | with XRPD peaks at |
| | | | approximately 8.0, 8.3, |
| | | | 12.1, 16.5, 17.7, 21.8, |
| | | | 23.1, 24.5, 27.1, and |
| | | | 28.4 degrees 2θ. |
| Crystalline | 2.5 | 1.84% | Crystalline Compound IA |
| Form XVI | | | with XRPD peaks at |
| | | | approximately 8.0, 8.3, |
| | | | 12.1, 16.5, 17.7, 21.8, |
| | | | 23.1, 24.5, 27.1, and |
| | | | 28.4 degrees 2θ. |
| Crystalline | 1.8 | 1.84% | Crystalline Compound IA |
| Form XVI | | | with XRPD peaks at |
| | | | approximately 8.0, 8.3, |
| | | | 12.1, 16.5, 17.7, 21.8, |
| | | | 23.1, 24.5, 27.1, and |
| | | | 28.4 degrees 2θ. |
| Crystalline | 1.8 | 5% | Crystalline Compound IA |
| Form XVI | | | with XRPD peaks at |
| | | | approximately 8.0, 8.3, |
| | | | 12.1, 16.5, 17.7, 21.8, |
| | | | 23.1, 24.5, 27.1, and |
| | | | 28.4 degrees 2θ. |
| Crystalline | 1.8 | 6% | Crystalline Compound IA |
| Form XVI | | | with XRPD peaks at |
| | | | approximately 8.0, 8.3, |
| | | | 12.1, 16.5, 17.7, 21.8, |
| | | | 23.1, 24.5, 27.1, and |
| | | | 28.4 degrees 2θ. |
| Crystalline | 1.8 | 8% | Crystalline Compound IA |
| Form XVI | | | with XRPD peaks at |
| | | | approximately 8.0, 8.3, |
| | | | 12.1, 16.5, 17.7, 21.8, |
| | | | 23.1, 24.5, 27.1, and |
| | | | 28.4 degrees 2θ. |
| Crystalline | 1.8 | 10% | Mixture of Compound I |
| Form XVI | | | free base and |
| | | | crystalline Compound |
| | | | IA with XRPD peaks at |
| | | | approximately 8.0, 8.3, |
| | | | 12.1, 16.5, 17.7, 21.8, |
| | | | 23.1, 24.5, 27.1, and |
| | | | 28.4 degrees 2θ. |
| Amorphous | 1.8 | 0.96% | Crystalline Compound IA |
| | | | with XRPD peaks at |
| | | | approximately 8.0, 8.3, |
| | | | 12.1, 16.5, 17.7, 21.8, |
| | | | 23.1, 24.5, 27.1, and |
| | | | 28.4 degrees 2θ. |
| Amorphous | 1.8 | 3.7% | Crystalline Compound IA |
| | | | with XRPD peaks at |
| | | | approximately 8.0, 8.3, |
| | | | 12.1, 16.5, 17.7, 21.8, |
| | | | 23.1, 24.5, 27.1, and |
| | | | 28.4 degrees 2θ. |
| Amorphous | 2.0 | 2.6% | Crystalline Compound IA |
| | | | with XRPD peaks at |
| | | | approximately 8.0, 8.3, |
| | | | 12.1, 16.5, 17.7, 21.8, |
| | | | 23.1, 24.5, 27.1, and |
| | | | 28.4 degrees 2θ. |
| Amorphous | 2.0 | 5.7% | Crystalline Compound IA |
| | | | with XRPD peaks at |
| | | | approximately 8.0, 8.3, |
| | | | 12.1, 16.5, 17.7, 21.8, |
| | | | 23.1, 24.5, 27.1, and |
| | | | 28.4 degrees 2θ. |
| Amorphous | 2.0 | 6.0% | Crystalline Compound IA |
| | | | with XRPD peaks at |
| | | | approximately 8.0, 8.3, |
| | | | 12.1, 16.5, 17.7, 21.8, |
| | | | 23.1, 24.5, 27.1, and |
| | | | 28.4 degrees 2θ. |
| Amorphous | 2.0 | 6.8% | Crystalline Compound IA |
| | | | with XRPD peaks at |
| | | | approximately 8.0, 8.3, |
| | | | 12.1, 16.5, 17.7, 21.8, |
| | | | 23.1, 24.5, 27.1, and |
| | | | 28.4 degrees 2θ. |
| Crystalline | 2.0 | 6.8% | Crystalline Compound IA |
| Form V | | | with XRPD peaks at |
| | | | approximately 8.0, 8.3, |
| | | | 12.1, 16.5, 17.7, 21.8, |
| | | | 23.1, 24.5, 27.1, and |
| | | | 28.4 degrees 2θ. |

### Example 3: Preparing Pure Crystalline Compound IA from Bulk Compound IA

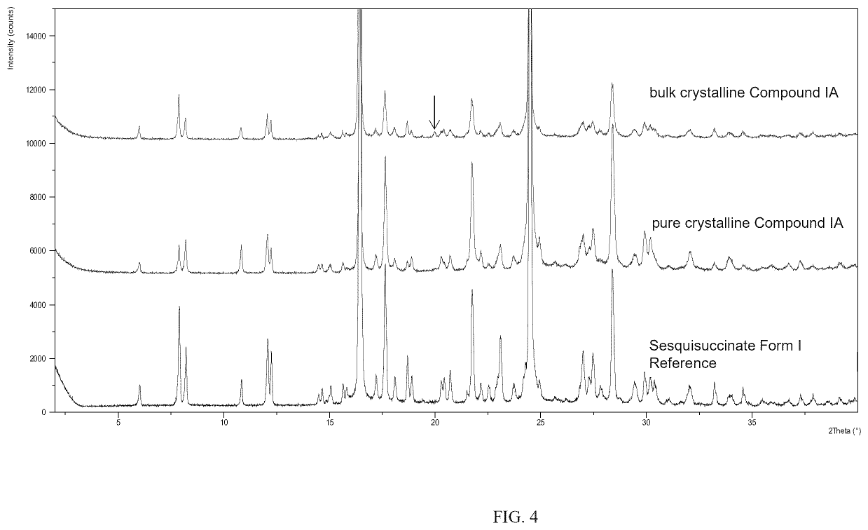

Crystalline Compound IA containing residual Compound I free base was processed into pure crystalline Compound I. 20 mg (0.03 mmol) of putative bulk crystalline Compound IA was treated with excess succinic acid (100 mg, 0.8 mmol; >20-fold excess of succinic acid) in 1 mL THF at 22° C. for 4 days. FIG. 4 shows that the putative bulk Compound IA (top XRPD) included a peak at about 19.9° 2θ corresponding to crystalline Compound I free base. After reprocessing, the peak at about 19.9° 2θ disappeared from the XRPD (middle XRPD), verifying conversion of the residual free base to the crystalline sesquisuccinate form. The bottom XRPD of FIG. 4, which is reference pure crystalline Compound IA, is included for comparison.

### X-Ray Powder Diffraction Crystal Preparation

Approximately 1 gram of the crystalline Compound IA was used. Existing solubility data was utilized for the design of experimentation. No specific storage precautions were indicated. Several single-crystals were obtained from the early experimentation and found to be the freebase (i.e., Compound I). The final experiment listed in the table is the one that successfully resulted in the single-crystal quality material. Crystalline Compound IA was weighed out (14.1 mg, 0.032 mmol) and placed into a 20 mL scintillation vial. To this was added approximately 3 mL of anhydrous THF, dried for 48 h over freshly activated 3 Å molecular sieves, resulting in a pale yellow suspension. To this solution was added 10.8 mg succinic acid (0.091 mmol, 3:1 ratio) under agitation. The suspension converted to a vibrant orange within five minutes. This was allowed to stir for 24 hours and was then filtered. The supernatant liquid was placed into an HPLC vial with a septum, which was punctured to allow controlled evaporation. Within 48 hours, yellow crystals suitable for single-crystal x-ray diffraction had formed.

Throughout this specification, various patents, patent applications and other types of publications (e.g., journal articles) are referenced. The disclosure of all patents, patent applications, and publications cited herein are hereby incorporated by reference in their entirety for all purposes.

1. A composition comprising crystalline Compound I sesquisuccinate, wherein the crystalline Compound I sesquisuccinate exhibits an X-ray powder diffraction pattern comprising at least three characteristic peaks, wherein said characteristic peaks are selected from the group consisting of approximately 8.0, 8.3, 12.1, 16.5, 17.7, 21.8, 23.1, 24.5, 27.1, and 28.4 degrees 2θ, and
wherein the crystalline Compound I sesquisuccinate in the composition comprises greater than about 75% of the total weight of the composition excluding pharmaceutically acceptable carriers.

2. (canceled)

3. The composition of claim 1, wherein an X-ray powder diffraction pattern of all Compound I in the composition does not contain a peak at approximately 19.9 degrees 2θ.

4. The composition of claim 1, wherein the crystalline Compound I sesquisuccinate has unit cell dimensions: a=8.370 Å; b=11.991 Å; c=15.605 Å; α=70.25°. β=82.14°; and γ=77.70°.

5. (canceled)

6. (canceled)

7. The composition of claim 1, wherein the composition comprises about 25 percent to about 30 percent by weight of succinate relative to the amount of Compound IA in the composition.

8. (canceled)

9. (canceled)

10. (canceled)

11. The composition of claim 1, wherein the crystalline Compound I sesquisuccinate in the composition comprises greater than about 85% of the total weight of the composition excluding pharmaceutically acceptable carriers.

12. (canceled)

13. (canceled)

14. (canceled)

15. (canceled)

16. (canceled)

17. (canceled)

18. (canceled)

19. A process for preparing crystalline Compound I sesquisuccinate, said process comprising the steps of:
(i) combining succinic acid and Compound I to form a crystallization mixture;
(ii) stirring the crystallization mixture; and
(iii) isolating a solid comprising Compound I sesquisuccinate from the crystallization mixture;
wherein at least 75% by weight of the solid isolated in step (iii) is crystalline Compound I sesquisuccinate, wherein the crystalline Compound I sesquisuccinate exhibits an X-ray powder diffraction pattern comprising at least three characteristic peaks, wherein said characteristic peaks are selected from the group consisting of approximately 8.0, 8.3, 12.1, 16.5, 17.7, 21.8, 23.1, 24.5, 27.1, and 28.4 degrees 2θ.

20. The process of claim 19, wherein the succinic acid is dissolved in a first solvent to form a first solution before combining said first solution with Compound I, wherein the first solvent is selected from the group consisting of tetrahydrofuran, 2-methyltetrahydrofuran, isopropyl alcohol, and acetone.

21. (canceled)

22. The process of claim 20, wherein the first solvent is tetrahydrofuran comprising from about 1% to 10% by weight water.

23. (canceled)

24. (canceled)

25. (canceled)

26. The process of claim 20, wherein the first solution is heated to a temperature of from about 35 to 45° C.

27. (canceled)

28. The process of claim 19, wherein the Compound I is dissolved in a second solvent to form a second solution before combining said second solution with succinic acid, wherein the second solvent is selected from the group consisting of tetrahydrofuran, 2-methyltetrahydrofuran, isopropyl alcohol, and acetone; and wherein the second solution is heated to a temperature of from about 35 to 45° C.

29. (canceled)

30. The process of claim 28, wherein the solvent is tetrahydrofuran comprising from about 1% to 10% by weight water.

31. (canceled)

32. (canceled)

33. (canceled)

34. (canceled)

35. (canceled)

36. The process of claim 28, wherein the second solution is added to the first solution to form the crystallization mixture; or wherein the first solution is added to the second solution to form the crystallization mixture.

37. (canceled)

38. (canceled)

39. (canceled)

40. (canceled)

41. (canceled)

42. The process of claim 19, wherein the crystallization mixture is stirred at a temperature of from about 35 to 45° C. for from about 8 to about 36 hours.

43. (canceled)

44. (canceled)

45. (canceled)

46. (canceled)

47. (canceled)

48. The process of claim 19, wherein a sample of precipitated solid is removed from the crystallization mixture and analyzed for succinic acid content, wherein the crystallization mixture is stirred for an additional 2 to 6 hours if said sample of precipitated solid is less than 27% by weight succinate.

49. (canceled)

50. The process of claim 19, wherein the Compound I in step (i) is amorphous; or wherein the Compound I in step (i) is a crystal form exhibiting an X-ray powder diffraction pattern comprising at least three characteristic peaks, wherein said characteristic peaks are selected from the group consisting of approximately 20.0, 18.0, 15.2, 10.8, 24.4, 20.8, 9.0, 19.4, and 17.3 degrees 2θ; or wherein the Compound I in step (i) is a crystal form exhibiting an X-ray powder diffraction pattern comprising at least three characteristic peaks, wherein said characteristic peaks are selected from the group consisting of 19.4, 23.3, 22.5, 16.7, 24.3, 13.9, 18.8, 18.3 and 21.1 degrees 2θ; or wherein the Compound I in step (i) is a crystal form exhibiting an X-ray powder diffraction pattern comprising at least three characteristic peaks, wherein said characteristic peaks are selected from the group consisting of 19.7, 22.7, 17.7, 14.0, 20.0, 8.6, 14.9, 21.3 and 17.2 degrees 2θ.

51. (canceled)

52. (canceled)

53. (canceled)

54. (canceled)

55. The process of claim 19, wherein greater than about 90% by weight of the solid isolated in step (iii) is crystalline Compound I sesquisuccinate.

56. (canceled)

57. (canceled)

58. (canceled)

59. (canceled)

60. (canceled)

61. (canceled)

62. The process of claim 19, wherein the molar ratio of Compound I to succinic acid combined in step (i) is from about 1:0.5 to about 1:5.

63. (canceled)

64. (canceled)

65. (canceled)

66. (canceled)

67. (canceled)

68. The process of claim 19, further comprising the step of filtering the crystallization mixture to recover wet Compound I sesquisuccinate; and wherein the wet Compound I sesquisuccinate is dried at a temperature of about 50° C. to about 70° C.

69. (canceled)

70. The process of claim 19, wherein an X-ray powder diffraction pattern of the isolated solid does not exhibit a peak at approximately 19.9 degrees 2θ.

71. (canceled)

72. (canceled)

73. A process for preparing substantially pure crystalline Compound I sesquisuccinate from a composition comprising one or more crystalline or amorphous forms of Compound I sesquisuccinate and optionally one or more crystalline or amorphous forms of Compound I, said process comprising the steps of:
(i) treating said composition with aqueous sulfuric acid to form Compound I sulfate; and
(ii) treating said Compound I sulfate with a base to form a first solution of Compound I; and
(iii) filtering said solution of Compound I to remove solids to form a second solution of Compound I.

74. (canceled)

75. (canceled)

76. (canceled)

77. (canceled)

78. (canceled)
